(19) United States
(12) Patent Application Publication
(10) Pub. No.: US 2009/0325150 A1
(43) Pub. Date: Dec. 31, 2009

(54) KLF6 ALTERNATIVE SPLICE FORMS AND A GERMLINE KLF6 DNA POLYMORPHISM ASSOCIATED WITH INCREASED CANCER RISK

(75) Inventors: John Martignetti, Chappaqua, NY (US); Goutham Narla, New Rochelle, NY (US); Scott Friedman, Scarsdale, NY (US)

Correspondence Address: DARBY & DARBY P.C., P.O. BOX 770, Church Street Station, New York, NY, 10008-0770, US

(73) Assignee: MOUNT SINAI SCHOOL OF MEDICINE OF NEW YORK UNIVERSITY, NEW YORK, NY (US)

(21) Appl. No.: 11/572,645

(22) PCT Filed: Aug. 1, 2005
(86) PCT No.: PCT/US05/27578

§ 371 Date: Jul. 6, 2009

Related U.S. Application Data
(60) Provisional application No. 60/592,782, filed on Jul. 30, 2004.
(60) Provisional application No. 60/601,304, filed on Aug. 13, 2004.

(51) Int. Cl. C12Q 1/68

(52) U.S. Cl. 435/6

(57) ABSTRACT

Disclosed are methods of identifying and diagnosing certain types of cancers and pre-stages thereof in a patient by identifying alternatively spliced isoforms of wild type KLF6 (KLFwt), in particular anyone of the isoforms selected from the group consisting of: KLF6 splice variant-1 (KLF6SV1), KLF6 splice variant-2 (KLF6SV2), and KLF6 splice variant-3 (KLF6SV3). Also disclosed are methods diagnosing cancer using the polypeptides and polynucleotides identified herein, as well as methods of treating certain types of cancers by inhibiting polynucleotides and polypeptides identified herein.

This application claims priority from U.S. Provisional Application Ser. No. 60/601,304, filed Aug. 13, 2004, and from U.S. Provisional Application Ser. No. 60/592,782, filed on Jul. 30, 2004, both of which are herein incorporated by reference in their entirety.

This application was funded, in part, by government grants R21-CA 84121, DAMD 17-02-1-0720, DAMD 17-03-1-0129, 2RO1DK37340-19, 2RO1DK56621-06, and DAMD17-03-1-0100. Accordingly, the government may have certain rights to this invention.

### FIELD OF THE INVENTION

The present invention relates to methods of treating and diagnosing certain types of cancer. In particular, the invention provides compositions comprising alternatively spliced isoforms of wild type KLF6, and to polynucleotides encoding such polypeptides. Such polypeptides and polynucleotides are useful in pharmaceutical compositions, and for screening for other compositions for the diagnosis and treatment of cancer.

### BACKGROUND OF THE INVENTION

### Cancer and Tumor Suppressors

Current cancer treatments such as surgery, chemotherapy, and radiation, remain less than completely effective for many patients. Gene therapy is providing new strategies for the treatments of malignant tumors. The advances that have been made over the past decade in the field of gene transfer, as well as in the fields of immunology and the molecular biology of tumorigenesis, have brought to reality the possibility of using gene transfer as an anti-cancer therapy.

The discovery of tumor suppressor genes has provided a new, genetic approach to the diagnosis, prevention and treatment of cancer. Research suggests that the replacement of the function of even a single pivotal gene can have significant anti-tumor effects. For example, the loss of the tumor suppressor retinoblastoma susceptibility gene (Rb) functions as a rate limiting event in the development of human and mouse cancers. Recent studies have demonstrated that correcting the Rb gene copy number can prevent carcinogenesis, and suppress neoplasia in mice (Nikitin, et al., Ann. NY Acad. Sci. 1999, 886:12 22). Additionally, preclinical studies in animal models have demonstrated that regression of tumors occurs following intratumoral administration of the p53 tumor suppressor gene (Reviewed in Roth, et al., Oncology. 1999, 10:148 54; Fujiwara, et al., Nippon Geka Gakkai Zasshi. 1999, 100:749 55). These results suggest that reconstitution of tumor suppressor function can be used as a method to treat cancer.

p53 is a tumor suppressor gene whose antitumor activity is in part related to its ability to upregulate the cell cycle inhibitor p21 in normal tissue. Thus, the loss of p53 in at least 50% of tumors leads to uncontrolled growth. However, up to 50% of human tumors have a mechanism of tumorigenesis other than defective p53 signaling. Thus, there is a need to identify other tumor suppressors that operate either independently from, or in conjunction with p53. Furthermore, there is a need in the art to understand how tumor suppressors become inactivated or less effective and the factors involved in this process. The silencing or inactivation of these factors, for example, can be used to treat cancer. In addition, there is a need in the art to identify oncogenes that work independently of tumor suppressor inactivation mechanisms to cause cancer.

Despite extensive research and advance, diagnosis of cancer in individuals has remained a difficult task to accomplish. Although some diagnostic markers are available that can be detected in blood or tissue samples, e.g. Carcinoembryonic Antigen (CEA), Alpha Fetoprotein (AFP) or Prostate Specific Antigen (PSA), assays using these markers have not, to date, been markedly predictive of the presence of cancer in these individuals, as verified by other clinical diagnoses. The sensitivity and specificity of these assays has been disappointingly low. Time consuming and labor intensive clinical assessments (e.g. palpations, x-rays, mammograms, biopsies) have remained the standard methods for diagnosing cancer. Thus, a need exists for a biomarker that is predictive of the presence of cancer, or of an increased risk of developing a cancer, in an individual. In particular, a need exists for a marker and an assay to measure the presence and amount of this marker for individuals with an early stage of cancer. If such a diagnostic test were available, early treatment with beneficial outcomes would be more likely than at present.

### Kruppel-Like Factor 6

The present invention addresses both therapeutic and diagnostic needs by disclosing novel KLF6 splice variants that are biologically active and can suppress wild-type (i.e., full-length) KLF6 tumor suppressor function. It has been discovered that the presence of KLF6 splice-variants are markers for the detection of certain type of cancer, and the prognosis (i.e., stage) of the cancer (including whether the cancer is likely to be or to become metastatic). Moreover, inhibition of the KLF6 splice-variants provides a treatment for tumors due to the oncogenic action of these splice variant proteins.

KLF6 is a gene encoding a novel zinc finger transcription factor protein. The gene was first cloned and reported as CPBP (Koritshconer, et al., Journal of Biol. Chem. 1997, 272, 9573 9580). While KLF6 was subsequently cloned from humans and rats as an immediate-early gene induced in hepatic stellate cells in early liver injury, it is expressed in all mammalian cell types (Ratziu, et al., Proc. Natl. Acad. Sci. USA 1998, 95:9500-9505). KLF6 has been localized to human chromosome 10p (Ratziu, et al., 1998, supra), a region which is deleted in various tumors including neuroblastomas, prostate cancer tumors, gliomas, and melanomas.

Nucleotide mutations and polymorphisms in wild-type KLF6 have previously been described (see U.S. patent application Ser. No. 10/752,079, to Friedman et al., filed Jan. 5, 2004), along with methods of diagnosis and prognosis by detecting such mutations or polymorphisms. However, the present invention also provides a novel mechanism of KLF6 inactivation in which a single nucleotide polymorphism (SNP) creates a novel splice site in the KLF6 gene, resulting in the production of KLF6 splice variants that inhibit or reduce the effectiveness of KLF6, thus preventing tumor-suppressor activity of KLF6. The present invention also discloses these KLF6 splice variants can form even in the absence of the SNP in the KLF6 gene, i.e., increases in the splice variants are present in tumors by a mechanism other than the SNP alteration. In addition, one of the splice variants promotes metastasis and tumorigenesis in addition to inhibiting wild type KLF6 activity. Furthermore, the present invention discloses that KLF6 splice variants can promote metastasis and tumorigenesis independently of wild type KLF6 activity.

### SUMMARY OF THE INVENTION

The present invention provides methods of identifying, and diagnosing, and determining the prognosis for certain types of cancers (including late-stage) and pre-stages thereof in a patient by identifying alternatively spliced isoforms of wild type KLF6 (KLF6_{wt}), in particular any one of the isoforms selected from the group consisting of: KLF6 splice variant-1 (KLF6_{SV1}), KLF6 splice variant-2 (KLF6_{SV2}), and KLF6 splice variant-3 (KLF6_{SV3}). The present invention further provides for methods of treating and preventing certain types of cancers using the polynucleotides and polypeptides identified herein. For example, the methods of this invention can be used to prevent cancer metastasis.

In one aspect, the present invention provides polynucleotide compositions comprising a sequence selected from the group consisting of: (a) sequences provided in SEQ ID NO: 1, SEQ ID NO: 3, and SEQ ID NO: 5; (b) complements of the sequences provided in SEQ ID NO: 1, SEQ ID NO: 3, and SEQ ID NO: 5; (c) sequences consisting of at least 20 contiguous residues of a sequence provided in SEQ ID NO: 1, SEQ ID NO: 3, and SEQ ID NO: 5; (d) sequences that hybridize to a sequence provided in SEQ ID NO: 1, SEQ ID NO: 3, and SEQ ID NO: 5, under normal hybridization conditions; and (e) sequences having at least 85% identity to a sequence of SEQ ID NO: 1, SEQ ID NO: 3, and SEQ ID NO: 5.

In another aspect, the present invention provides polypeptide compositions comprising an amino acid sequence that is encoded by a polynucleotide sequence described above. Additionally, the present invention further provides polypeptide compositions comprising an amino acid sequence selected from the group consisting of sequences recited in SEQ ID NO: 2, SEQ ID NO: 4, and SEQ ID NO: 6.

In a further aspect, the present invention provides polynucleotides that encode a polypeptide described above, expression vectors comprising such polynucleotides and host cells transformed or transfected with such expression vectors.

In another embodiment, the present invention provides antibodies, such as monoclonal antibodies, and in a particular embodiment, humanized monoclonal antibodies, that bind to any one of the KLF6 variant polypeptides described above. In certain embodiments, the antibodies of the present invention may be used in diagnostic kits for the diagnosis and stratified classification of cancer. In additional embodiments the antibodies of the present invention may be used in pharmaceutical compositions useful in the treatment and prevention of certain cancers.

In other aspects, the present invention provides methods for determining the progression of cancer in a subject diagnosed with a tumor by determining the presence of any one of the KLF6 splice variants of the present invention. In certain embodiments of the present invention, the method of determining the progression of cancer in a subject comprises the determining the ratio between wild-type KLF6 and a KLF6 splice variant, wherein a decrease in the ratio of wild-type KLF6 to a KLF6 splice variant relative to the ratio of wild-type KLF6 to a KLF6 splice measured on a previous occasion or relative to the ratio in a non-cancerous control is indicative of the progression of cancer. In a specific embodiment, the KLF6 variant is KLF6_{SV1}.

Within other aspects, the present invention provides pharmaceutical compositions comprising an agent capable of modulating the activity of any one of the KLF6 splice variants, and a pharmaceutically acceptable carrier. In a particular embodiment, the present invention provides a pharmaceutical composition comprising (i) an antibody or antigen-binding fragment thereof that specifically binds to a polypeptide of the present invention, or an immunological fragment thereof; and (ii) a pharmaceutically acceptable carrier. In yet another embodiment, the invention provides a pharmaceutical composition comprising (i) an interfering RNA nucleic acid, and (ii) a pharmaceutically acceptable carrier.

Within a further aspect, the present invention provides methods for inhibiting the progression and/or metastasis of a cancer in a patient, comprising the steps of administering to a patient a pharmaceutical composition as recited above. The patient may be afflicted with a cancer, in which case the methods provide treatment for the disease, or a patient considered to be at risk for such a disease may be treated prophylactically.

The present invention also provides screening methods for identifying inhibitors for the KLF6 splice variants which can be used for the treatment of cancer.

These and other aspects of the present invention will become apparent upon reference to the following detailed description, attached drawings and non-limiting examples. All references disclosed herein are hereby incorporated by reference in their entirety.

### BRIEF DESCRIPTION OF THE DRAWINGS

FIG. 1 shows structure and cellular localization of KLF6 and its splice variants. Genomic organization, splice site sequences, and mRNA structures for wtKLF6 and KLF6 splice variants are illustrated. NLS, nuclear localization signal. PCR primer binding sites for qtRT-PCR are shown.

FIG. 2(a-e) shows the expression of KLF6 and its splice variants in prostate cancer. (a) DNA microarray analysis of KLF6 mRNA in hormone-refractory metastatic prostate cancer (HR-MET) as compared to both normal prostate tissue and localized prostate cancer; NAP, normal adjacent prostate tissue; PCA, localized prostate cancer. (b) Tissue microarray analysis of KLF6 expression using a KLF6 monoclonal antibody (2A2). The mean KLF6 protein expression is summarized using error bars with 95% confidence intervals for both localized prostate cancer (localized PCa) and metastatic disease (MET) (c) RT-PCR of representative prostate derived cDNAs with KLF6 specific primers; N, normal prostate, L, localized prostate cancer, M, metastatic prostate cancer. (d) qtRT-PCR analysis of localized and metastatic prostate cancer cDNAs for both wild type KLF6 expression and KLF6 alternative splicing. (e) KLF6_{SV1} expression in metastatic prostate cancer; western blot using the KLF6 2A2 antibody; M, metastatic disease, B, benign prostatic hyperplasia, wt, transfected wtKLF6, SV1, transfected KLF6_{SV1}, L, localized prostate cancer.

FIG. 3 shows relative splicing in matched non-tumor (N) and tumorous (T) liver from 7 liver explants with HCC.

FIG. 4(a-b) shows cytoplasmic accumulation of KLF6 in cirrhosis and HCC (a and b, respectively). Both panels are from the same patient.

FIG. 5(a-b) shows (a) qtRT-PCR analysis of KLF6 alternative splicing in tumor cells transfected with siRNA prior to injection; and (b) qtRT-PCR analysis of KLF6 alternative splicing in cells treated with siRNA after injection.

FIG. 6(a-b) shows the divergent effects of stably expressed siRNAs to KLF6 wt and SV1, respectively on PC3M xenograft growth in vivo (a) and direct intratumoral injection of the pSUPER-si-SV1 plasmid in a prostate cancer xenograft model (b).

FIG. 7(a-b) shows the divergent effects of stably expressed siRNAs to wt KLF6 and KLF6_{SV1} on VEGF secretion in cultured cells and in vivo VEGF mRNA and protein levels in stable SKOV3 cell lines expressing siRNAs to either luciferase, wild type KLF6, or KLF6_{SV1}. Changes in VEGF message (a) and the 189 and 165 secreted amino acid isoforms (b).

FIG. 8(a-c) shows migration and invasion following abrogation of KLF6_{SV1} by siRNA in PC3M cells (a) Comparison of cell migration in si-SV1 and si-luc treated cells; (b) Comparison of invasion in si-SV1 and si-luc treated cells; (c) Decreasing wtKLF6 expression by siRNA results in increased tumor invasion and intraperitoneal growth. Upper panels demonstrate representative photomicrographs of the underside of a matrigel insert stained with DAPI. Lower panels express these findings as number of cells/400× magnified visual field.

FIG. 9(a-f) shows (a) Results of qtRTPCR using total KLF6 and wtKLF6 primers on cells transfected with either the wtKLF6 or IVSΔA minigene construct. (Middle Panel b,c,d left to right) (b) Western blot analysis of BPH1 cells transfected with IVSΔA and wtKLF6 minigene constructs using a KLF6 polyclonal antibody. (c) Stable expression of the IVSΔA variant in BPH1 results in an increase of KLF6 splicing. (d) Western blot analysis of BPH1 stable cell lines stably expressing the IVSΔA or wtKLF6 minigene construct. (e) qtRT-PCR from benign prostatic hyperplasia cell line (BPH1) and the metastatic prostate cancer cell line (PC3M) transfected with a KLF6 full length minigene construct. (f) Western blot analysis for KLF6 expression in BPH1 and PCM cells.

### DETAILED DESCRIPTION OF THE INVENTION

The present invention is, in part, based on the identification of alternatively spliced, biologically active KLF6 isoforms (which, in part, have dominant-negative roles towards wild type KLF6 tumor suppressive functions) and are associated with certain types of cancer, including hormone-dependent cancers. In particular, the invention contemplates diagnosis of cancers characterized by the increased expression and/or activity of KLF6 splice variants. Accordingly, the invention provides nucleotide sequences encoding alternatively spliced KLF6_{wt} isoforms associated with cancer and pre-stages thereof. Such isoforms include, but not limited to, KLF6_{SV1}, KLF6_{SV2}, and KLF6_{SV3}. The alternatively spliced KLF6_{wt} isoforms are characterized by, for example, their ability to decrease KLF6_{wt} functionality, i.e., loss-of-function in KLF6_{wt} activity or by, for example, a decrease in p21^{(CIP1/Waf1)} expression levels, as compared to controls non-cancerous cells. Characterization of such cancers provides a basis for therapy, prognosis, or diagnosis, as detailed below.

### KLF6 Splice Variant Polynucleotide Compositions

The present invention provides polynucleotide compositions. As used here the terms “polynucleotide,” “nucleotide sequence,” and “nucleic acid sequence” are interchangeable and mean series of nucleotide bases in DNA and RNA, and mean any chain of two or more nucleotides. A nucleotide sequence typically carries genetic information, including the information used by cellular machinery to make proteins and enzymes. These terms include double or single stranded genomic and cDNA, RNA, any synthetic and genetically manipulated polynucleotides, and both sense and anti-sense polynucleotides (although only sense stands are being represented herein). This includes single- and double-stranded molecules, i.e., DNA-DNA, DNA-RNA and RNA-RNA hybrids, as well as “protein nucleic acids” (PNAs) formed by conjugating bases to an amino acid backbone. This also includes nucleic acids containing modified bases, for example thio-uracil, thio-guanine and fluoro-uracil.

As will be understood by those skilled in the art, the polynucleotide compositions of this invention can include genomic sequences, extra-genomic and plasmid-encoded sequences and smaller engineered gene segments that express, or may be adapted to express, proteins, polypeptides, peptides and the like. Such segments may be naturally isolated, or modified synthetically by the hand of man.

As also will be recognized by the skilled artisan, polynucleotides of the invention may be single-stranded (coding or antisense) or double-stranded, and may be DNA (genomic, cDNA or synthetic) or RNA molecules. RNA molecules may include heterogeneous nuclear RNA (hnRNA) molecules that contain an internal transcribed poly A sequence and introns and correspond to a DNA molecule in a one-to-one manner, as well as mRNA molecules, which do not contain introns. Other RNA molecules include inhibitory RNA molecules (RNAi; siRNA, shRNA; ribozymes and triple-helix).

Polynucleotides may comprise a native sequence (i.e., an endogenous sequence that encodes a polypeptide/protein of the invention or a portion thereof) or may comprise a sequence that encodes a variant or derivative, preferably an immunogenic variant or derivative, of such a sequence.

Therefore, according to another aspect of the present invention, polynucleotide compositions are provided that comprise some or all of a polynucleotide sequence set forth in any one of SEQ ID NO: 1, SEQ ID NO: 3, or SEQ ID NO: 5, as well as complements of a polynucleotide sequence set forth in any one of SEQ ID NO: 1, SEQ ID NO: 3, or SEQ ID NO: 5. The present invention also embodies degenerate variants of a polynucleotide sequence set forth in any one of SEQ ID NO: 1, SEQ ID NO: 3, or SEQ ID NO: 5.

In other embodiments, the present invention provides for isolated polynucleotides encoding splice variants of wild-type KLF6 (“KLF6_{wt}”), herein referred to as “KLF6 splice variants,” including, but not limited to the polynucleotide encoding KLF6 splice variant-1, referred to herein as “KLF6_{SV1},” having the polynucleotide sequence of SEQ ID NO: 1, KLF6 splice variant-2, referred to herein as “KLF6_{SV2},” having the polynucleotide sequence of SEQ ID NO: 3, and KLF6 splice variant-3, referred to herein as “KLF6_{SV3},” having the polynucleotide sequence of SEQ ID NO: 5.

As used herein, the term “isolated” means that the referenced material is removed from the environment in which it is normally found. Thus, an isolated biological material can be free of cellular components, i.e., components of the cells in which the material is found or produced. In the case of nucleic acid molecules, an isolated nucleic acid includes a PCR product, an isolated mRNA, a cDNA, or a restriction fragment. In another embodiment, an isolated nucleic acid is preferably excised from the chromosome in which it may be found, and more preferably is no longer joined to non-regulatory, non-coding regions, or to other genes, located upstream or downstream of the gene contained by the isolated nucleic acid molecule when found in the chromosome. In yet another embodiment, the isolated nucleic acid lacks one or more introns. Isolated nucleic acid molecules include sequences inserted into plasmids, cosmids, artificial chromosomes, and the like. Thus, in a specific embodiment, a recombinant nucleic acid is an isolated nucleic acid. An isolated protein may be associated with other proteins or nucleic acids, or both, with which it associates in the cell, or with cellular membranes if it is a membrane-associated protein. An isolated organelle, cell, or tissue is removed from the anatomical site in which it is found in an organism. An isolated material may be, but need not be, purified.

The term “purified” as used herein refers to material that has been isolated under conditions that reduce or eliminate the presence of unrelated materials, i.e., contaminants, including native materials from which the material is obtained. For example, a purified protein is preferably substantially free of other proteins or nucleic acids with which it is associated in a cell; a purified nucleic acid molecule is preferably substantially free of proteins or other unrelated nucleic acid molecules with which it can be found within a cell. As used herein, the term “substantially free” is used operationally, in the context of analytical testing of the material. Preferably, purified material substantially free of contaminants is at least 50% pure; more preferably, at least 90% pure, and more preferably still at least 99% pure. Purity can be evaluated by chromatography, gel electrophoresis, immunoassay, composition analysis, biological assay, and other methods known in the art.

Methods for purification are well-known in the art. For example, nucleic acids can be purified by precipitation, chromatography (including preparative solid phase chromatography, oligonucleotide hybridization, and triple helix chromatography), ultracentrifugation, and other means. Polypeptides and proteins can be purified by various methods including, without limitation, preparative disc-gel electrophoresis, isoelectric focusing, HPLC, reversed-phase HPLC, gel filtration, ion exchange and partition chromatography, precipitation and salting-out chromatography, extraction, and countercurrent distribution. For some purposes, it is preferable to produce the polypeptide in a recombinant system in which the protein contains an additional sequence tag that facilitates purification, such as, but not limited to, a polyhistidine sequence, or a sequence that specifically binds to an antibody, such as FLAG and GST. The polypeptide can then be purified from a crude lysate of the host cell by chromatography on an appropriate solid-phase matrix. Alternatively, antibodies produced against the protein or against peptides derived therefrom can be used as purification reagents. Cells can be purified by various techniques, including centrifugation, matrix separation (e.g., nylon wool separation), panning and other immunoselection techniques, depletion (e.g., complement depletion of contaminating cells), and cell sorting (e.g., fluorescence activated cell sorting [FACS]). Other purification methods are possible. A purified material may contain less than about 50%, preferably less than about 75%, and most preferably less than about 90%, of the cellular components with which it was originally associated. The “substantially pure” indicates the highest degree of purity which can be achieved using conventional purification techniques known in the art.

In other related embodiments, the present invention provides polynucleotide variants are substantially identical to any one of SEQ ID NO: 1, SEQ ID NO: 3, or SEQ ID NO: 5, for example those comprising at least about 70% sequence identity, preferably at least about 85%, more preferably at least 90% and still more preferably at least about 95% or higher, sequence identity compared to a polynucleotide sequence of this invention using the methods described herein. One skilled in the art will recognize that these values can be appropriately adjusted to determine corresponding identity of proteins encoded by two nucleotide sequences by taking into account codon degeneracy, amino acid similarity, reading frame positioning and the like.

In preferred embodiments, the terms “about” and “approximately” shall generally mean an acceptable degree of error for the quantity measured given the nature or precision of the measurements. Typical, exemplary degrees of error are within 20 percent (%), preferably within 10%, and more preferably within 5% of a given value or range of values. Alternatively, and particularly in biological systems, the terms “about” and “approximately” may mean values that are within an order of magnitude, preferably within 5-fold and more preferably within 2-fold of a given value. Numerical quantities given herein are approximate unless stated otherwise, meaning that the term “about” or “approximately” can be inferred when not expressly stated.

When comparing polynucleotide sequences, two sequences are said to be “identical” if the sequence of nucleotides in the two sequences is the same when aligned for maximum correspondence, as described below. Comparisons between two sequences are typically performed by comparing the sequences over a comparison window to identify and compare local regions of sequence similarity. A “comparison window” as used herein, refers to a segment of at least about 20 contiguous positions, usually about 30 to about 75, preferably about 40 to about 50, in which a sequence may be compared to a reference sequence of the same number of contiguous positions after the two sequences are optimally aligned.

Optimal alignment of sequences for comparison may be conducted using any one of the optimal alignment methods known in the art, for example using the local identity algorithm of Smith and Waterman, Add. APL. Math 1981; 2: 482, by the identity alignment algorithm of Needleman and Wunsch, J. Mol. Biol. 1970; 48: 443, by the search for similarity methods of Pearson and Lipman, Proc. Natl. Acad. Sci. USA. 1988; 85: 2444, by computerized implementations of these algorithms (GAP, BESTFIT, BLAST, FASTA, and TFASTA in the Wisconsin Genetics Software Package, Genetics Computer Group (GCG), Madison, Wis.), or by inspection.

One preferred example of algorithms that are suitable for determining percent sequence identity and sequence similarity are the BLAST and BLAST 2.0 algorithms, which are described in Altschul et al. Nucl. Acids Res. 1977; 25: 3389-3402 and Altschul et al. J. Mol. Biol. 1990; 215: 403-410, respectively. BLAST and BLAST 2.0 can be used, for example with the parameters described herein, to determine percent sequence identity for the polynucleotides of the invention. Software for performing BLAST analyses is publicly available through the National Center for Biotechnology Information.

Preferably, the “percentage of sequence identity” is determined by comparing two optimally aligned sequences over a window of comparison of at least 20 positions, wherein the portion of the polynucleotide sequence in the comparison window may comprise additions or deletions (i.e., gaps) of 20 percent or less, usually 5 to 15 percent, or 10 to 12 percent, as compared to the reference sequences (which does not comprise additions or deletions) for optimal alignment of the two sequences. The percentage is calculated by determining the number of positions at which the identical nucleic acid bases occurs in both sequences to yield the number of matched positions, dividing the number of matched positions by the total number of positions in the reference sequence (i.e., the window size) and multiplying the results by 100 to yield the percentage of sequence identity.

It will be appreciated by those of ordinary skill in the art that, as a result of the degeneracy of the genetic code, there are many nucleotide sequences that encode a polypeptide as described herein. Some of these polynucleotides bear minimal homology to the nucleotide sequence of any native gene. Nonetheless, polynucleotides that vary due to differences in codon usage are specifically contemplated by the present invention. Further, alleles of the genes comprising the polynucleotide sequences provided herein are within the scope of the present invention. Alleles are endogenous genes that are altered as a result of one or more mutations, such as deletions, additions and/or substitutions of nucleotides. The resulting mRNA and protein may, but need not, have an altered structure or function. Alleles may be identified using standard techniques (such as hybridization, amplification and/or database sequence comparison).

In another embodiment of the invention, polynucleotide compositions are provided that are capable of hybridizing under normal conditions to a polynucleotide sequence provided herein, or a fragment thereof, or a complementary sequence thereof. Hybridization techniques are well known in the art of molecular biology. A nucleic acid molecule is “hybridizable” to another nucleic acid molecule, such as a cDNA, genomic DNA, or RNA, when a single stranded form of the nucleic acid molecule can anneal to the other nucleic acid molecule under the appropriate conditions of temperature and solution ionic strength (see, Sambrook et al., Molecular Cloning: A Laboratory Manual, Third Edition (2001) Cold Spring Harbor Laboratory Press, Cold Spring: New York, herein referred to as “Sambrook et al. (2001)”). Hybridization requires that the two nucleic acids contain complementary sequences, although depending on the stringency of the hybridization, mismatches between bases are possible. The conditions of temperature and ionic strength determine the “stringency” of the hybridization. The appropriate stringency for hybridizing nucleic acids depends on the length of the nucleic acids and the degree of complementation, variables well known in the art. The greater the degree of similarity or homology between two nucleotide sequences, the greater the value of Tₘ for hybrids of nucleic acids having those sequences. The relative stability (corresponding to higher Tₘ) of nucleic acid hybridizations decreases in the following order: RNA:RNA, DNA:RNA, DNA:DNA. A minimum length for a hybridizable nucleic acid is at least about 10 nucleotides; preferably at least about 15 nucleotides; and more preferably the length is at least about 20 nucleotides.

In a specific embodiment, the term “normal hybridization conditions” refers hybridization and/or washing conditions at, for example, 5×SSC, 0.1% SDS, 0.25% milk, and no formamide; or 30% formamide, 5×SSC, 0.5% SDSm. Wherein Tₘ is 55° C. In a preferred embodiment, the Tₘ is 60° C.; in a more preferred embodiment, the Tₘ is 65° C. In a specific embodiment, “high stringency” refers to hybridization and/or washing conditions at 68° C. in 0.2×SSC, at 42° C. in 50% formamide, 4×SSC, or under conditions that afford levels of hybridization equivalent to those observed under either of these two conditions.

In other embodiments of the present invention, the polynucleotide sequences provided herein can be used as probes or primers for nucleic acid hybridization. As such, it is contemplated that nucleic acid segments that comprise a sequence region of at least about 15 contiguous nucleotides that has the same sequence as, or is complementary to, a 15 nucleotide long contiguous sequence disclosed herein will find particular utility. Longer contiguous identical or complementary sequences, e.g., those of about 20, 30, 40, 50, 100, 200 (including all intermediate lengths) and even up to full length sequences may be useful as probes or primers for nucleic acid hybridization.

Polynucleotide molecules having sequence regions consisting of contiguous nucleotide stretches from about 10 to about 19, from about 20 to about 29, and from about 30 to about 50, or even of 100-200 nucleotides or so (including intermediate lengths as well), identical or complementary to a polynucleotide sequence disclosed herein, are particularly contemplated as hybridization probes for use in, e.g., Southern and Northern blotting. This would allow a gene product, or fragment thereof, to be analyzed, both in diverse cell types and also in various bacterial cells. The total size of fragment, as well as the size of the complementary stretch(es), will ultimately depend on the intended use or application of the particular nucleic acid segment. Smaller fragments will generally find use in hybridization embodiments, wherein the length of the contiguous complementary region may be varied, such as between about 15 and about 100 nucleotides, but larger contiguous complementarily stretches may be used, according to the length complementary sequences one wishes to detect.

Hybridization probes may be selected from any portion of any of the sequences disclosed herein. All that is required is to review the sequences set forth herein, or to any continuous portion of the sequences, from about 15-25 nucleotides in length up to and including the full length sequence, that one wishes to utilize as a probe or primer.

Small polynucleotide segments or fragments may be readily prepared by, for example, directly synthesizing the fragment by chemical means, as is commonly practiced using an automated oligonucleotide synthesizer. Also, fragments may be obtained by application of nucleic acid reproduction technology, such as the polymerase chain reaction (“PCR”), as described in U.S. Pat. No. 4,683,202, which is hereby incorporated by reference, by introducing selected sequences into recombinant vectors for recombinant production, and by other recombinant DNA techniques generally known to those of skill in the art of molecular biology.

### KLF6 Splice Variant Polypeptide Compositions

As used herein, the term “polypeptide” as used herein, retains its conventional meaning, i.e., as a sequence of amino acids. The polypeptides are not limited to a specific length of the product; thus, peptides, oligopeptides, and proteins are included within the definition of polypeptide, and such terms may be used interchangeably herein unless specifically indicated otherwise. This term also does not refer to or exclude post-expression modifications of the polypeptide, for example, glycosylations, acetylations, phosphorylations and the like, as well as other modifications known in the art, both naturally occurring and non-naturally occurring. A polypeptide may be an entire protein, or a subsequence thereof.

Particularly illustrative polypeptides of the present invention comprise those encoded by a polynucleotide sequence set forth in any one of SEQ ID NO: 1, SEQ ID NO: 3, or SEQ ID NO: 5 or a degenerate variant thereof. The term “degenerate variants” of a polynucleotide sequence are those in which a change of one or more nucleotides in a given codon position results in no alteration in the amino acid encoded at that position. The present invention also provides polypeptides encoded by a polynucleotide sequence that hybridizes under normal hybridization conditions as set forth above to any one of SEQ ID NO: 1, SEQ ID NO: 3, or SEQ ID NO: 5. In specific embodiments, the polypeptides of the invention comprise amino acid sequences as set forth in any one of SEQ ID NO: 2, SEQ ID NO: 4, or SEQ ID NO: 6 or a degenerate variant thereof.

In other embodiments, the present invention provides KLF6 splice variants including, but not limited to, KLF6_{SV1} having the amino acid sequence of SEQ ID NO: 2, KLF6_{SV2} having the amino acid sequence of SEQ ID NO: 4, and KLF6_{SV3} having the amino acid sequence of SEQ ID NO: 6.

The present invention, in another aspect, provides polypeptide fragments comprising at least about 5, 10, 15, 20, 25, 50, or 100 contiguous amino acids, or more, including all intermediate lengths, of a polypeptide composition set forth herein, such as those set forth in SEQ ID NO: 2, SEQ ID NO: 4, and SEQ ID NO: 6, or those encoded by a polynucleotide sequence set forth in a sequence of any one of SEQ ID NO: 1, SEQ ID NO: 3, and SEQ ID NO: 5.

In another aspect, the present invention provides variants of the polypeptide compositions described herein. Polypeptide variants generally encompassed by the present invention will typically exhibit at least about 70% sequence identity, preferably at least about 85%, more preferably at least 90% and still more preferably at least about 95% or higher or more identity (determined as described above for determining identity of polynucleotide sequences), along its length, to a polypeptide sequence set forth herein.

A polypeptide “variant,” as used herein, is a polypeptide that typically differs from a polypeptide specifically disclosed herein in one or more substitutions, deletions, additions and/or insertions. Such variants may be naturally occurring or may be synthetically generated, for example, by modifying one or more of the above polypeptide sequences of the invention and evaluating their immunogenic activity as described herein using any of a number of techniques well known in the art.

For example, certain illustrative variants of the polypeptides of the invention include those in which one or more portions, such as an N-terminal leader sequence or transmembrane domain, have been removed. Other illustrative variants include variants in which a small portion (e.g., 1-30 amino acids, preferably 5-15 amino acids) has been removed from the N- and/or C-terminal of the mature protein. Further variants include polypeptides in which a peptide tag has been added to the N- and/or C-terminus of the protein to, for example, improve protein purification yields. Tags include but are not limited to galactosidase, maltose-binding protein fusions, glutathione-S-transferase, polyhistidine fusions, V5, HA, myc, and FLAG.

In many instances, a variant will contain conservative substitutions. A “conservative substitution” is one in which an amino acid is substituted for another amino acid that has similar properties, such that one skilled in the art of peptide chemistry would expect the secondary structure and hydropathic nature of the polypeptide to be substantially unchanged. As described above, modifications may be made in the structure of the polynucleotides and polypeptides of the present invention and still obtain a functional molecule that encodes a variant or derivative polypeptide with desirable characteristics, e.g., with immunogenic characteristics.

For example, certain amino acids may be substituted for other amino acids in a protein structure without appreciable loss of interactive binding capacity with structures such as, for example, antigen-binding regions of antibodies or binding sites on substrate molecules. Since it is the interactive capacity and nature of a protein that defines that protein's biological functional activity, certain amino acid sequence substitutions can be made in a protein sequence, and, of course, its underlying DNA coding sequence, and nevertheless obtain a protein with like properties. It is thus contemplated that various changes may be made in the peptide sequences of the disclosed compositions, or corresponding DNA sequences which encode said peptides without appreciable loss of their biological utility or activity.

The present invention further provides immunogenic portions of the polypeptides disclosed herein. An “immunogenic portion,” as used herein, is a fragment of an immunogenic polypeptide of the invention that itself is immunologically reactive (i.e., specifically binds) with the B-cells and/or T-cell surface antigen receptors that recognize the polypeptide. Immunogenic portions may generally be identified using well known techniques, such as those summarized in Harlow and Lane 1988. Such techniques include screening polypeptides for the ability to react with antigen-specific antibodies, antisera and/or T-cell lines or clones. As used herein, antisera and antibodies are “antigen-specific” if they specifically bind to an antigen (i.e., they react with the protein in an ELISA or other immunoassay, and do not react detectably with unrelated proteins). Such antisera and antibodies may be prepared as described herein, and using well-known techniques.

### Polynucleotide Identification, Expression and Characterization

The polynucleotides of the present invention may be isolated from a sample and identified using any one of the well established molecular biology methods, such as those described in Sambrook, et al. 2001 and Herndon and Rychlik, In White, B. A. (ed.), Methods in Molecular Biology. 1993; Vol. 15, pp 31-39, PCR Protocols: Current Methods and Applications, Humania Press, Inc., Totowa, N.J. Such methods include template based methods, such as the polymerase chain reaction (“PCR”), which is described in detail in U.S. Pat. Nos. 4,683,195, 4,683,202 and 4,800,159, each of which is hereby incorporated by reference in its entirety.

The polynucleotides of the present invention may be isolated from a sample using PCR by using at least one oligonucleotide primer that is substantially complementary to only one of the strands in the target. A primer refers to an oligonucleotide that can be extended with a DNA polymerase using monodeoxyribonucleoside triphosphates and a nucleic acid that is used as a template. This primer preferably has a 3′ hydroxyl group on an end that is facing the 5′ end of the template nucleic acid when it is hybridized with the template.

In another embodiment, the polynucleotides of the present invention may be isolated using PCR using a pair of oligonucleotide primers. A set of primers refers to a combination or mixture of at least a first (forward) and a second (reverse) primer. The first primer can be extended using the template nucleic acid while forming an extension product in such a way that the second primer can hybridize with this extension product in a region of the extension product that lies in the 3′ direction of the extendable end of the first primer. The extendable end of the second primer points in the 5′ direction of the extension product of the first primer. Examples of primers that are suitable for performing PCR and that meet this definition are described in European Patent Application No. 0201184, which is hereby incorporated by reference. Typical amplicons range in size from 25 bp to 2000 bp (see, e.g., U.S. Pat. No. 6,518,025). Larger sized amplicons can be obtained, typically using specialized conditions or modified polymerases.

The primers of the present invention are designed to be specific (i.e., hybridizes to) regions of the polynucleotides identified herein. Primers contemplated include nucleic acid segments that comprise a sequence region of at least about 15 contiguous nucleotides that has the same sequence as, or is complementary to, a 15 nucleotide long contiguous sequence disclosed herein. Longer contiguous identical or complementary sequences, e.g., those of about 20, 30, 40, 50, 100, 200 (including all intermediate lengths) and even up to full length sequences may be useful as probes or primers for nucleic acid hybridization. Particularly useful primers include, but are not limited to, those having the polynucleotide sequence of any one of SEQ ID NO: 9, 10, 13-16.

Following amplification, the products of PCR may be detected using any one of a variety of PCR detection methods are known in the art including standard non-denaturing gel electrophoresis (e.g., acrylamide or agarose), denaturing gradient gel electrophoresis, and temperature gradient gel electrophoresis. Standard non-denaturing gel electrophoresis is the simplest and quickest method of PCR detection, but may not be suitable for all applications.

Also contemplated by the present invention are any one of a number of other template dependent processes known and available in the art. Such methods include, but are not limited to, ligase chain reaction (“LCR”), described, for example, in U.S. Pat. No. 4,883,750; Qbeta Replicase, described in International Patent Application No. PCT/US87/00880; Strand Displacement Amplification (“SDA”) and Repair Chain Reaction (RCR).

An amplified portion of a polynucleotide of the present invention may be used to isolate a full length gene from a suitable library (e.g., a tumor cDNA library) using well known techniques. Within such techniques, a library (cDNA or genomic) is screened using one or more polynucleotide probes or primers suitable for amplification. Preferably, a library is size-selected to include larger molecules. Random primed libraries may also be preferred for identifying 5′ and upstream regions of genes. Genomic libraries are preferred for obtaining introns and extending 5′ sequences.

For hybridization techniques, a partial sequence may be labeled (e.g., by nick-translation or end-labeling with P32) using well known techniques. A bacterial or bacteriophage library is then generally screened by hybridizing filters containing denatured bacterial colonies (or lawns containing phage plaques) with the labeled probe (see e.g., Sambrook et al. 2001, supra). Hybridizing colonies or plaques are selected and expanded, and the DNA is isolated for further analysis. cDNA clones may be analyzed to determine the amount of additional sequence by, for example, PCR using a primer from the partial sequence and a primer from the vector. Restriction maps and partial sequences may be generated to identify one or more overlapping clones. The complete sequence may then be determined using standard techniques, which may involve generating a series of deletion clones. The resulting overlapping sequences can then assembled into a single contiguous sequence. A full length cDNA molecule can be generated by ligating suitable fragments, using well known techniques.

Alternatively, amplification techniques, such as those described above, can be useful for obtaining a full length coding sequence from a partial cDNA sequence. One such amplification technique is inverse PCR (see Triglia et al., Nucl. Acids Res. 1988; 16: 8186), which uses restriction enzymes to generate a fragment in the known region of the gene. A variation on this procedure, which employs two primers that initiate extension in opposite directions from the known sequence, is described in WO 96/38591. Another technique is known as “rapid amplification of cDNA ends” or RACE. Additional techniques include capture PCR (Lagerstrom et al., PCR Methods Applic. 1991; 1: 111-19) and walking PCR (Parker et al., Nucl. Acids. Res. 1991; 19: 3055-60). Other methods employing amplification may also be employed to obtain a full length cDNA sequence.

In additional embodiments, the present invention provides an expression vector comprising any one of the polynucleotides provided herein, particularly polynucleotides encoding KLF6 splice variants, immunogenic fragment or functionally active fragments thereof. The expression vector of the present invention contains the necessary elements for the transcription and translation of the inserted protein-coding sequence. Thus, a polynucleotide encoding any one of the KLF6 splice variants of the invention can be operationally associated with a promoter in an expression vector of the invention. Both cDNA and genomic sequences can be cloned and expressed under control of such regulatory sequences. Such vectors can be used to express functional or functionally inactivated KLF6 splice variant.

The necessary transcriptional and translational signals can be provided on a recombinant expression vector. Potential host-vector systems include but are not limited to mammalian cell systems transfected with expression plasmids or infected with virus (e.g., vaccinia virus, adenovirus, adeno-associated virus, herpes virus, etc.); insect cell systems infected with virus (e.g., baculovirus); microorganisms such as yeast containing yeast vectors; or bacteria transformed with bacteriophage, DNA, plasmid DNA, or cosmid DNA. The expression elements of vectors vary in their strengths and specificities. Depending on the host-vector system utilized, any one of a number of suitable transcription and translation elements may be used.

Expression of a KLF6 splice variant polypeptide of the present invention may be controlled by any promoter/enhancer element known in the art, but these regulatory elements must be functional in the host selected for expression. Promoters which may be used to control expression of any one of the polynucleotides of the present invention include, but are not limited to, cytomegalovirus (CMV) promoter (U.S. Pat. Nos. 5,385,839 and 5,168,062), the SV40 early promoter region (Benoist and Chambon, Nature. 1981; 290: 304-310), the promoter contained in the 3′ long terminal repeat of Rous sarcoma virus (Yamamoto, et al., Cell. 1980; 22: 787-797), the herpes thymidine kinase promoter (Wagner et al., Proc. Natl. Acad. Sci. U.S.A. 1981; 78: 1441-1445), the regulatory sequences of the metallothionein gene (Brinster et al., Nature. 1982; 296: 39-42); prokaryotic expression vectors such as the β-lactamase promoter (VIIIa-Komaroff et al., Proc. Natl. Acad. Sci. U.S.A. 1978; 75: 3727-3731), or the tac promoter (DeBoer et al., Proc. Natl. Acad. Sci. U.S.A. 1983; 80: 21-25). Still other useful promoter elements which may be used include promoter elements from yeast or other fungi such as the Gal 4 promoter, the ADC (alcohol dehydrogenase) promoter, PGK (phosphoglycerol kinase) promoter, alkaline phosphatase promoter; and transcriptional control regions that exhibit hematopoietic tissue specificity, in particular: beta-globin gene control region which is active in myeloid cells (Mogram et al., Nature. 1985; 315: 338-340), hematopoietic stem cell differentiation factor promoters, erythropoietin receptor promoter (Maouche et al., Blood. 1991; 15: 2557).

A wide variety of host/expression vector combinations may be employed in expressing the DNA sequences of this invention. Useful expression vectors, for example, may consist of segments of chromosomal, non chromosomal and synthetic DNA sequences. Suitable vectors include derivatives of SV40 and known bacterial plasmids, e.g., E. coli plasmids col El, pCR1, pBR322, pMa1-C2, pET, pGEX (Smith et al., Gene. 1988; 67: 31-40), pCR2.1 and pcDNA 3.1+(Invitrogen, Carlsbad, Calif.), pMB9 and their derivatives, plasmids such as RP4; phage DNAs, e.g., the numerous derivatives of phage 1, e.g., NM989, and other phage DNA, e.g., M13 and filamentous single stranded phage DNA; yeast plasmids such as the 2m plasmid or derivatives thereof; vectors useful in eukaryotic cells, such as vectors useful in insect or mammalian cells; vectors derived from combinations of plasmids and phage DNAs, such as plasmids that have been modified to employ phage DNA or other expression control sequences; and the like.

### KLF6 Splice Variant Antibodies

According to another aspect, the present invention further provides binding agents, such as antibodies and antigen-binding fragments thereof, that exhibit immunological binding to any one of the KLF6 splice variants disclosed herein, or to a portion, variant or derivative thereof. An antibody, or antigen-binding fragment thereof, is said to “specifically bind,” “immunogically bind,” and/or is “immunologically reactive” to a polypeptide of the invention if it reacts at a detectable level (within, for example, an ELISA assay) with the polypeptide, and does not react detectably with unrelated polypeptides under similar conditions. Such antibodies include, but are not limited to, polyclonal, monoclonal, chimeric, single chain, Fab fragments, and Fab expression library.

Various procedures known in the art may be used for the production of polyclonal antibodies to any one of the KLF6 splice variants or derivative or analog thereof. For the production of antibody, various host animals can be immunized by injection with the antigenic polypeptide, including but not limited to rabbits, mice, rats, sheep, goats, etc.

For preparation of monoclonal antibodies directed toward the any one of the KLF6 splice variants described herein, any technique that provides for the production of antibody molecules by continuous cell lines in culture may be used. These include but are not limited to the hybridoma technique originally developed by Kohler and Milstein (see, e.g., Nature. 1975; 256: 495-497), as well as the trioma technique, the human B-cell hybridoma technique (Kozbor et al., Immunology Today. 1983; 4: 72; Cote et al., Proc. Natl. Acad. Sci. U.S.A. 1983; 80: 2026-2030), and the EBV-hybridoma technique to produce human monoclonal antibodies (Cole et al., in Monoclonal Antibodies and Cancer Therapy, Alan R. Liss, Inc., pp. 77-96, (1985)). In an additional embodiment of the invention, monoclonal antibodies can be produced in germ-free animals (see, e.g., International Patent Publication No. WO 89/12690).

The present invention further provides humanized monoclonal antibodies that exhibit immunological binding to any one of the KLF6 splice variants disclosed herein. A number of “humanized” antibody molecules comprising an antigen-binding site derived from a non-human immunoglobulin have been described, including chimeric antibodies having rodent V regions and their associated complementarily-determining regions (“CDRs”) fused to human constant domains (see, e.g., Winter et al. Nature. 1991; 349: 293-299), and rodent CDRs grafted into a human supporting FR prior to fusion with an appropriate human antibody constant domain (see, e.g., Riechmann et al., Nature. 1988; 332: 323-327; Verhoeyen et al., Science. 1988; 239:1534-1536). These “humanized” antibodies are designed to minimize unwanted immunological response toward rodent antihuman antibody molecules which limits the duration and effectiveness of therapeutic applications of those moieties in human recipients.

According to the invention, techniques described for the production of single chain antibodies (see e.g., U.S. Pat. Nos. 5,476,786, 5,132,405 and 4,946,778) can be adapted to produce the KLF6 splice variant-specific single chain antibodies. Indeed, these genes can be delivered for expression in vivo. An additional embodiment of the invention utilizes the techniques described for the construction of Fab expression libraries (Huse et al., Science. 1989; 246:1275-1281) to allow rapid and easy identification of monoclonal Fab fragments with the desired specificity for a any one of the KLF6 splice variant, or its derivatives, or analogs.

Antibody fragments which contain the idiotype of the antibody molecule can be generated by known techniques. For example, such fragments include but are not limited to: the F(ab′)2 fragment which can be produced by pepsin digestion of the antibody molecule; the Fab′ fragments which can be generated by reducing the disulfide bridges of the F(ab′)2 fragment, and the Fab fragments which can be generated by treating the antibody molecule with papain and a reducing agent.

In the production of antibodies, screening for the desired antibody can be accomplished by techniques known in the art, e.g., radioimmunoassay, ELISA (enzyme-linked immunosorbant assay), “sandwich” immunoassays, immunoradiometric assays, gel diffusion precipitin reactions, immunodiffusion assays, in situ immunoassays (using colloidal gold, enzyme or radioisotope labels, for example), western blots, precipitation reactions, agglutination assays (e.g., gel agglutination assays, hemagglutination assays), complement fixation assays, immunofluorescence assays, protein A assays, and immunoelectrophoresis assays, etc. In one embodiment, antibody binding is detected by detecting a label on the primary antibody. In another embodiment, the primary antibody is detected by detecting binding of a secondary antibody or reagent to the primary antibody. In a further embodiment, the secondary antibody is labeled. Many means are known in the art for detecting binding in an immunoassay and are within the scope of the present invention.

### Detection and Diagnosis of Cancer Using KLF6 Splice Variants

In other aspects the present invention provides diagnostic methods for the determination of the presence of cancer in a patient. Cancer may be detected in a patient, preferably a human patient, based upon the presence of any one of the polynucleotide and/or polypeptides in a biological sample from the patient. As used herein, “diagnostic methods” include both diagnostic and prognostic methods, i.e., methods of providing a prognosis of potential therapeutic outcome or severity of the cancer.

In another embodiment, the compositions described herein may be used as markers for the progression of cancer, wherein the term “progression of cancer” refers to cancer cell generation, proliferation, metastasis, or a worsening of tumor grade. In this embodiment, assays as described below for the diagnosis of a cancer may be performed over time, and the change in the level of reactive polypeptide(s) or polynucleotide(s) evaluated. For example, the assays may be performed repeatedly about every 24 to 72 hours for a period of about 6 months to about 1 year, and thereafter performed as needed. In general, a cancer is progressing in those patients in whom the difference between the level of polypeptide or polynucleotide detected in the patient and the level of polypeptide or polynucleotide in a non-cancerous control changes over time. In contrast, the cancer is not progressing when the level of reactive polypeptide or polynucleotide either remains constant or the difference changes with time.

In yet another aspect of the present invention, the detection and diagnostic methods described herein may be adapted to assess the severity of cancer by determining the ratio of KLF6_{wt} to any one of the KLF6 splice variants described herein, wherein a low ratio of KLF6_{wt} to KLF6 splice variant is indicative of an advanced stage of cancer and/or an advanced tumor stage. In particular embodiments, the ratio of KLF6_{wt} to KLF6 splice variant is determined using any one of the detection methods described below. In one preferred embodiment the severity of cancer is evaluated by determining the ratio of KLF6_{wt} to KLF6_{SV1}, wherein an increasing ratio of KLF6_{SV1} to KLF6_{wt} is indicative of an advanced stage of cancer.

As used herein, the term “diagnosis” refers to detecting the presence of a disease or disorder, including determining the presence of the disease at any stage of its development. This term also includes the determination of a predisposition of a subject to develop the disease, i.e., determining the likelihood of developing a disease in an individual at risk.

The diagnostic method of the invention encompasses, but not restricted to, screening individuals for the potential of different types of cancers such as solid phase tumors/malignancies, locally advanced tumors, human soft tissue sarcomas, metastatic cancer, including lymphatic metastases, blood cell malignancies including multiple myeloma, acute and chronic leukemias, and lymphomas, head and neck cancers including mouth cancer, larynx cancer and thyroid cancer, lung cancers including small cell carcinoma and non-small cell cancers, breast cancers including small cell carcinoma and ductal carcinoma, gastrointestinal cancers including esophageal cancer, stomach cancer, colon cancer, colorectal cancer and polyps associated with colorectal neoplasia, pancreatic cancers, liver cancer, urologic cancers including bladder cancer and prostate cancer, malignancies of the female genital tract including ovarian carcinoma, uterine (including endometrial) cancers, and solid tumor in the ovarian follicle, kidney cancers including renal cell carcinoma, brain cancers including intrinsic brain tumors, neuroblastoma, astrocytic brain tumors, gliomas, metastatic tumor cell invasion in the central nervous system, bone cancers including osteomas, skin cancers including malignant melanoma, tumor progression of human skin keratinocytes, squamous cell carcinoma, basal cell carcinoma, hemangiopericytoma and Karposi's sarcoma.

“Prognosis” refers to predicting the course or severity of the disease or condition, e.g., the likelihood of worsening of the disease with and without treatment. For example, if the disease or condition is associated with a solid phase tumor/malignancy, there is a better prognosis if therapy to prevent and/or inhibit tumor growth is instituted.

The term “biological sample” refers to any cell source from which DNA may be obtained. Non-limiting examples of cell sources available in clinical practice include without limitation blood cells, buccal cells, cervicovaginal cells, epithelial cells from urine, fetal cells, or any cells present in tissue obtained by biopsy. Cells may also be obtained from body fluids, including without limitation blood, plasma, serum, lymph, milk, cerebrospinal fluid, saliva, sweat, urine, feces, and tissue exudates (e.g., pus) at a site of infection or inflammation. For prenatal testing, genetic material can be obtained from fetal cells, e.g., from amniotic fluid (through amniocentesis), chronic villi, blood, or any tissue of a pregnant woman. DNA is extracted using any of the numerous methods that are standard in the art. It will be understood that the particular method used to extract DNA will depend on the nature of the source. Generally, the minimum amount of DNA to be extracted for use in the present invention is about 25 pg (corresponding to about 5 cell equivalents of a genome size of 4×10⁹ base pairs). Various methods for detecting the polynucleotides and polypeptides of the present invention are described herein.

### Nucleic Acid Based Detection and Diagnosis Methods

In certain embodiments, the diagnostic methods of the invention encompass detecting any one of the polynucleotides provided herein and in particular, mRNA encoding any one of the KLF6 splice variants of the present invention, such as KLF6_{SV1}, KLF6_{SV2}, or KLF6_{SV3}. The level of mRNA in a biological sample may be determined using a PCR based assay, by amplifying a portion of cDNA derived from a biological sample using a pair of oligonucleotide primers, wherein at least one of the oligonucleotide primers is specific for (i.e., hybridizes to) a polynucleotide encoding one of the KLF6 splice variants of the present invention. Following amplification, the amplified cDNA may be separated and detected using techniques well known in the art, such as gel electrophoresis. The instant diagnostic method contemplates that the presence of an amplification product is indicative of cancer.

In one preferred embodiment the diagnostic method employs reverse transcription-PCR (“RT-PCR”). The diagnostic method comprises the first step of extracting total RNA from a biological sample, and performing reverse transcription to produce cDNA molecules. PCR amplification is than performed using a pair of oligonucleotide primers, wherein at least one of the oligonucleotide primers is specific for a polynucleotide encoding one of the KLF6 splice variants of the present invention. Particularly preferred primers for use in the PCR amplification step include: SEQ ID NOS: 9, 10, and 13-16.

Amplification may be performed on biological samples taken from a test patient and from an individual who is not afflicted with a cancer. The amplification reaction may be performed on several dilutions of cDNA spanning two orders of magnitude. About at least a 20% increase in expression in several dilutions of the test patient sample as compared to the same dilutions of the non-cancerous sample is typically considered positive.

Similarly, oligonucleotide probes that specifically hybridize to a polynucleotide encoding one of the KLF6 splice variants of the present invention may be used in a hybridization assay to detect the presence of the polynucleotide of interest in a biological sample. To permit hybridization under assay conditions, oligonucleotide probes should comprise an oligonucleotide sequence that has at least about 60%, preferably at least about 75% and more preferably at least about 90%, identity to a portion of a polynucleotide encoding one of the KLF6 splice variants of the invention that is at least 10 nucleotides, and preferably at least 20 nucleotides, in length. Preferably, probes hybridize to a polynucleotide encoding a polypeptide described herein under moderately stringent conditions, as defined above. Oligonucleotide probes which may be usefully employed in the diagnostic methods described herein preferably are at least 10-40 nucleotides in length. In a preferred embodiment, the oligonucleotide primers comprise at least 10 contiguous nucleotides, more preferably at least 15 contiguous nucleotides, of a DNA molecule having a sequence as disclosed herein.

### Antibody Detection and Diagnosis Methods

In other aspects, the invention provides for methods of detecting and diagnosing cancer by immunoassay. In particular, the antibodies described above may be useful in the immunoassays disclosed herein. Immunoassays include, for example, Western blotting which permits detection of any one of the KLF6 splice variants of the present invention. Other immunoassay formats, such as those described above for the production of antibodies can also be used in place of Western blotting. These include, for example, ELISA assays as described in Harlow and Lane (1988).

In ELISA assays, an antibody against any one of the KLF6 splice variants of the present invention, or an epitopic fragment thereof, is immobilized onto a selected surface, for example, a surface capable of binding proteins such as the wells of a polystyrene microtiter plate. After washing to remove incompletely adsorbed polypeptides, a nonspecific protein such as a solution of bovine serum albumin (BSA) may be bound to the selected surface. This allows for blocking of nonspecific adsorption sites on the immobilizing surface and thus reduces the background caused by nonspecific bindings of antisera onto the surface. The immobilizing surface is then contacted with a sample, to be tested in a manner conductive to immune complex (antigen/antibody) formation. This may include diluting the sample with diluents, such as solutions of BSA, bovine gamma globulin (BGG) and/or phosphate buffered saline (PBS)/Tween. The sample is then allowed to incubate for from 2 to 4 hours, at temperatures between about 25° to 37° C. Following incubation, the sample-contacted surface is washed to remove non-immunocomplexed material. The washing procedure may include washing with a solution, such as PBS/Tween or borate buffer. Following formation of specific immunocomplexes between the test sample and the bound antibody, and subsequent washing, the occurrence, and an even amount of immunocomplex formation may be determined by subjecting the immunocomplex to a second antibody against any one of the KLF6 splice variants that recognizes a different epitope on the protein. To provide detecting means, the second antibody may have an associated activity such as an enzymatic activity that will generate, for example, a color development upon incubating with an appropriate chromogenic substrate. Quantification may then be achieved by measuring the degree of color generation using, for example, a visible spectra spectrophotometer.

Typically the detection antibody is conjugated to an enzyme such as peroxidase and the protein is detected by the addition of a soluble chromophore peroxidase substrate such as tetramethylbenzidine followed by 1 M sulfuric acid. The test protein concentration is determined by comparison with standard curves.

Alternatively, a biochemical assay can be used to detect expression, or accumulation of any one of the KLF6 splice variants of the present invention, e.g., by detecting the presence or absence of a band in samples analyzed by polyacrylamide gel electrophoresis; by the presence or absence of a chromatographic peak in samples analyzed by any of the various methods of high performance liquid chromatography, including reverse phase, ion exchange, and gel permeation; by the presence or absence of any one of the KLF6 splice variants in analytical capillary electrophoresis chromatography, or any other quantitative or qualitative biochemical technique known in the art.

### Diagnostic Kits

The invention also provides kits for performing the diagnostic methods described herein. A particular subject of the invention is a kit for diagnosing different types of cancers, comprising an oligonucleotide that specifically hybridizes to any one of the polynucleotides described herein, and in particular a polynucleotide encoding any one of the KLF6 splice variants of the present invention. In a particular embodiment, the probe may hybridize to any one of the polynucleotide sequences of SEQ ID NO: 1, SEQ ID NO: 3, or SEQ ID NO: 5. A further subject of the invention is a kit for diagnosing a cancer, comprising an antibody that specifically recognizes any one of the KLF6 splice variants of the present invention, including for example, the KLF6 splice variants having the amino acid sequence of SEQ ID NO: 2, SEQ ID NO: 4, or SEQ ID NO: 6.

### Nucleic Acid Based Diagnostic Kits

The invention provides nucleic acid-based methods for detecting any one of the KLF6 splice variants in a biological sample. The presence of a polynucleotide encoding any one of the KLF6 splice variants may be determined using any suitable means known in the art, including without limitation one or more of hybridization with specific probes for PCR amplification, restriction fragmentation, direct sequencing, SSCP, and other techniques known in the art.

The present invention also provides kits suitable for nucleic acid-based diagnostic applications. In one embodiment, diagnostic kits include the following components: (i) probe DNA and (ii) hybridization reagents. The probe DNA may be pre-labeled; alternatively, the probe DNA may be unlabeled and the ingredients for labeling may be included in the kit in separate containers. The kit may also contain other suitably packaged reagents and materials needed for the particular hybridization protocol, including solid-phase matrices, if applicable, and standards.

In another embodiment, diagnostic kits include: (i) sequence determination primers and (ii) sequence determination reagents. The sequencing primers may be pre-labeled or may contain an affinity purification or attachment moiety. The kit may also contain other suitably packaged reagents and materials needed for the particular sequencing protocol.

### Antibody Based Diagnostic Kits

The invention also provides antibody-based methods for detecting the KLF6 splice variants of the present invention in a biological sample. The methods comprise the steps of: (i) contacting a sample with one or more antibody preparations, wherein each of the antibody preparations is specific for any one of the KLF6 splice variants described herein, under conditions in which a stable antigen-antibody complex can form between the antibody and the KLF6 splice variant in the sample; and (ii) detecting any antigen-antibody complex formed in step (i) using any suitable means known in the art, wherein the detection of a complex indicates the presence of the KLF6 splice variant.

Typically, immunoassays use either a labeled antibody or a labeled antigenic component (e.g., that competes with the antigen in the sample for binding to the antibody). Suitable labels include without limitation enzyme-based, fluorescent, chemiluminescent, radioactive, or dye molecules. Assays that amplify the signals from the probe are also known, such as, for example, those that utilize biotin and avidin, and enzyme-labeled immunoassays, such as ELISA assays.

The present invention also provides kits suitable for antibody-based diagnostic applications. Diagnostic kits typically include one or more of the following components: (i) KLF6 splice variant-specific antibodies, and (ii) reaction components. The antibodies may be pre-labeled; alternatively, the antibody may be unlabeled and the ingredients for labeling may be included in the kit in separate containers, or a secondary, labeled antibody is provided. The kit may also contain other suitably packaged reagents and materials needed for the particular immunoassay protocol, including solid-phase matrices, if applicable, and standards.

The kits referred to above may include instructions for conducting the test. Furthermore, in preferred embodiments, the diagnostic kits are adaptable to high-throughput and/or automated operation.

### Methods of Prevention and Treatment of Cancer

The present invention further provides a method treating cancer. In certain embodiments the method of treating cancer provided herein comprises reducing and/or preventing the progression of cancer, wherein the term “progression of cancer” refers to cancer cell generation, proliferation, metastasis, or a worsening of tumor grade. Methods of detecting and diagnosing the progression of cancer are described above.

Without being bound by any particular theory, the method of treating cancer provided herein promotes tumor regression, e.g., reduces tumor growth and/or proliferation, by inhibiting tumor cell proliferation, inhibiting angiogenesis (growth of new blood vessels that is necessary to support tumor growth) and/or inhibiting or reducing metastasis by reducing tumor cell motility or invasiveness. The methods of prevention and treatment provided herein may be effective in adult and pediatric oncology including in solid phase tumors/malignancies, locally advanced tumors, human soft tissue sarcomas, metastatic cancer, including lymphatic metastases, blood cell malignancies including multiple myeloma, acute and chronic leukemias, and lymphomas, head and neck cancers including mouth cancer, larynx cancer and thyroid cancer, lung cancers including small cell carcinoma and non-small cell cancers, breast cancers including small cell carcinoma and ductal carcinoma, gastrointestinal cancers including esophageal cancer, stomach cancer, colon cancer, colorectal cancer and polyps associated with colorectal neoplasia, pancreatic cancers, liver cancer, urologic cancers including bladder cancer and prostate cancer, malignancies of the female genital tract including ovarian carcinoma, uterine (including endometrial) cancers, and solid tumor in the ovarian follicle, kidney cancers including renal cell carcinoma, brain cancers including intrinsic brain tumors, neuroblastoma, astrocytic brain tumors, gliomas, metastatic tumor cell invasion in the central nervous system, bone cancers including osteomas, skin cancers including malignant melanoma, tumor progression of human skin keratinocytes, squamous cell carcinoma, basal cell carcinoma, hemangiopericytoma and Karposi's sarcoma.

In certain additional embodiments, the method of treating cancer comprises modulating the activity of any one of the KLF6 splice variants of the present invention, in a subject or patient. The method comprises administering to a patient in need of such treatment an effective amount of an agent that modulates the activity of any one of the KLF6 splice variants of the present invention, with a pharmaceutically acceptable carrier. For example, the therapeutic agent may be a antisense polynucleotide, double stranded RNA species mediating RNA interference (“RNAi”), or an intracellular inhibitory antibody which specifically binds any one of the KLF6 splice variants of the present invention.

A “subject” or “patient” is a human or an animal with, or likely to develop, a cancer, more particularly a mammal, preferably a rodent or a primate, as described above in connection with diagnostic applications.

The term “treatment” means to therapeutically intervene in the development of a disease in a subject showing a symptom of this disease. The term “treatment” also encompasses prevention, which means to prophylactically interfere with a pathological mechanism that results in the disease.

The term “modulating the activity of any one of the KLF6 splice variants” in a subject refers to increasing or decreasing the expression of the splice variant nucleic acid or polypeptide, or reducing or inhibiting an activity of the splice varian polypeptide. As on e example, this term encompasses both inhibiting the functionality of any one of the KLF6 splice variants, as well as inhibiting, preventing or blocking the expression of KLF6 splice variants in a subject and in one preferred embodiment, in a subject having a cancer. As another example, “modulating the activity of any one of the KLF6 splice variants” in a subject also can mean preventing and/or altering its ability to bind KLF6_{wt}, as well as affecting its ability to prevent the migration of KLF6_{wt} into the nucleus of a cell.

The modulation of the activity of any one of the KLF6 splice variants of the present invention may be achieved by various methods, as described hereafter.

In one embodiment, the modulatory agent may be a substance that is known or has been identified to modulate, especially inhibit, whether fully or partially, the activity of any one of the KLF6 splice variants of the present invention. Such compounds can include any compound(s) described herein. The modulatory agent also may be a candidate drug as identified by a screening method. It may also be an inhibitory antibody directed against any one of the KLF6 splice variants. In a further embodiment, it may be an antisense nucleic acid. In an additional embodiment the modulatory agent may be a double stranded RNA species mediating RNA interference. All these embodiments are described in greater detail below.

The term “therapeutically effective amount” is used herein to mean an amount or dose sufficient to modulate, e.g., decrease the level of KLF6 splice variant activity by about 10 percent, preferably by about 50 percent, and more preferably by about 90 percent. Preferably, a therapeutically effective amount can ameliorate or present a clinically significant deficit in the activity, function and response of the subject. Alternatively, a therapeutically effective amount is sufficient to cause an improvement in a clinically significant condition in the host, i.e. cancer.

The substance that modulates or inhibits KLF6 splice variant activity is advantageously formulated in a pharmaceutical composition, with a pharmaceutically acceptable carrier. The concentration or amount of the active ingredient depends on the desired dosage and administration regimen, as discussed below. Suitable dose ranges may include from about 0.01 mg/kg to about 100 mg/kg of body weight per day. The pharmaceutical compositions may also include other biologically active compounds.

The phrase “pharmaceutically acceptable” refers to molecular entities and compositions that are physiologically tolerable and do not typically produce an allergic or similar untoward reaction, such as gastric upset, dizziness and the like, when administered to a human. Preferably, as used herein, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant, excipient, or vehicle with which the compound is administered. Such pharmaceutical carriers can be sterile liquids, such as water and oils, including those of petroleum, animal, vegetable or synthetic origin, such as peanut oil, soybean oil, mineral oil, sesame oil and the like. Water or aqueous solution saline solutions and aqueous dextrose and glycerol solutions are preferably employed as carriers, particularly for injectable solutions. Suitable pharmaceutical carriers are described in “Remington's Pharmaceutical Sciences” by E. W. Martin.

According to the invention, the pharmaceutical composition of the invention can be introduced parenterally, transmucosally, e.g., orally, nasally, or rectally, or transdermally. Parenteral routes include local and systemic routes and include intravenous, intra-arteriole, intramuscular, intradermal, subcutaneous, intraperitoneal; intraventricular, intratumoral, intrathecal and intracranial administration. In particular, for tumors of the brain (e.g., gliomas), or of the head and neck, it may be necessary to employ local administration such as intrathecal or intracranial (via e.g., a cannula) for administering antisense or siRNA, since these molecules do not cross the blood brain barrier. Alternatively, a variety of techniques are available for CNS administration which promote transfer of the therapeutic agent across the blood brain barrier, including disruption by surgery or injection, co-administration of a drug that transiently opens adhesion contacts between CNS vasculature endothelial cells, and co-administration of a substance that facilitates translocation through such cells.

In addition, the pharmaceutical composition of the present invention can be administered

The pharmaceutical compositions may be added to a retained physiological fluid such as blood or synovial fluid.

In another embodiment, the active ingredient can be delivered in a vesicle, in particular a liposome (see, e.g., Langer, Science 1990; 249: 1527-1533 and Treat et al., in Liposomes in the Therapy of Infectious Disease and Cancer, Lopez-Berestein and Fidler (eds.), Liss:New York, pp. 353-365 (1989)).

In yet another embodiment, the therapeutic compound can be delivered in a controlled release system. For example, a polypeptide may be administered using intravenous infusion with a continuous pump, in a polymer matrix such as poly-lactic/glutamic acid (PLGA), a pellet containing a mixture of cholesterol and the active ingredient (SilasticR™; Dow Corning, Midland, Mich.; see U.S. Pat. No. 5,554,601) implanted subcutaneously, an implantable osmotic pump, a transdermal patch, liposomes, or other modes of administration.

### Inhibition of SR Protein Binding

In one embodiment, the activity of any one of the KLF6 splice variants of the present invention may be modulated by administering an agent that inhibits the binding of a Serine-Arginine (SR) protein such as, for example SRp20, SRp30, SRp40, SRp55, or SRp75, to a polynucleotide encoding the KLF6 gene having a single nucleotide polymorphism −27 G>A (referred to herein as the “novel binding site”-set forth in SEQ ID NO: 19). In a particularly preferred embodiment the activity of a KLF6 splice variant is inhibited by administering an agent that inhibits the binding of SRp40 to the novel binding site.

Agents that inhibit the binding of a SR protein to the IVSΔA allele include, but are not limited to, anti-SR protein antibodies, siRNA to the SR protein message, small chimeric effectors comprising a minimal synthetic RS domain (see, e.g., Cartegni L., and Krainer A. R.; Nat Struct Biol. 2003; 10:120-5), and agents that disrupt splice protein selection and/or use such as those described in Muraki M., et al., J Biol Chem. 2004; 279: 24246-54. Other agents include those that out-compete binding of an SR protein such as SRp40 to the novel binding site (e.g., SEQ ID NO: 19). Such agents include ‘decoy’ nucleic acid sequences comprising the SR protein novel binding sites. Alternatively, other agents that bind to the novel binding site in lieu of an SR protein, but do not cause formation of a KLF6 splice variant (i.e., by alternative splicing of wild-type KLF6), can be administered in excess to block binding of the SR protein to endogenous KLF6 novel binding site. Methods of screening for binding of agents to nucleic acids (i.e., nucleic acids comprising the novel binding site of SEQ ID NO: 19) are routine and well-known in the art.

### Inhibitory Antibodies

The modulatory substance may also be an antibody that is directed against any one of the KLF6 splice variants of the present invention. Antibodies that block the activity of any one of the KLF6 splice variants may be produced and selected according to any standard method well-known by one skilled in the art, such as those described above in the context of diagnostic applications.

Intracellular antibodies (sometime referred to as “intrabodies”) have been used to regulate the activity of intracellular proteins in a number of systems (see, Marasco, Gene Ther. 1997; 4: 11; Chen et al., Hum. Gene Ther. 1994; 5: 595), e.g., viral infections (Marasco et al., Hum. Gene Ther. 1999; 9: 1627) and other infectious diseases (Rondon et al., Annu. Rev. Microbiol. 1997; 51: 257), and oncogenes, such as p21 (Cardinale et al., FEBS Lett. 1998; 439: 197-202; Cochet et al., Cancer Res. 1998; 58: 1170-6), myb (Kasono et al., Biochem Biophys Res Commun. 1998; 251: 124-30), erbB-2 (Graus-Porta et al., Mol Cell Biol. 1995; 15: 1182-91). This technology can be adapted to inhibit the activity of KLF6 splice variants by expression of an antibody that is reactive against any one the KLF6 splice variants, e.g., an anti-KLF6 splice variant antibody.

In a particularly preferred embodiment intracellular antibodies are monoclonal antibodies and more preferably they are humanized monoclonal antibodies. Examples of preferred antibodies include, but are not limited to anti-KLF6_{SV1}, anti-KLF6_{SV2}, and anti-KLF6_{SV3}, and particularly preferred antibodies include those prepared as described below, including the monoclonal antibody that specifically binds KLF6 KLF6_{SV1} (SEQ ID NO: 4) and is designated antibody 9A2.

In another embodiment of the invention, monoclonal antibodies of the present invention may be coupled to one or more therapeutic agents. Suitable agents in this regard include radionuclides, differentiation inducers, drugs, toxins, and derivatives thereof. Preferred radionuclides include Y⁹⁰, I¹²³, I²⁵, 186Re¹⁸⁶, Re¹⁸⁸, At²¹¹, and Bi²¹². Preferred drugs include methotrexate, and pyrimidine and purine analogs. Preferred differentiation inducers include phorbol esters and butyric acid. Preferred toxins include ricin, abrin, diptheria toxin, cholera toxin, gelonin, Pseudomonas exotoxin, Shigella toxin, and pokeweed antiviral protein.

A therapeutic agent may be coupled (e.g., covalently bonded) to a suitable antibody either directly or indirectly (e.g., via a linker group). A direct reaction between an agent and an antibody is possible when each possesses a substituent capable of reacting with the other. For example, a nucleophilic group, such as an amino or sulfhydryl group, on one may be capable of reacting with a carbonyl-containing group, such as an anhydride or an acid halide, or with an alkyl group containing a good leaving group (e.g., a halide) on the other.

Alternatively, it may be desirable to couple a therapeutic agent and an antibody via a linker group. A linker group can function as a spacer to distance an antibody from an agent in order to avoid interference with binding capabilities. A linker group can also serve to increase the chemical reactivity of a substituent on an agent or an antibody, and thus increase the coupling efficiency. An increase in chemical reactivity may also facilitate the use of agents, or functional groups on agents, which otherwise would not be possible.

It will be evident to those skilled in the art that a variety of bifunctional or polyfunctional reagents, both homo- and hetero-functional (such as those commercially available from Pierce Chemical Co., Rockford, Ill.), may be employed as the linker group. Coupling may be effected, for example, through amino groups, carboxyl groups, sulfhydryl groups or oxidized carbohydrate residues. There are numerous references describing such methodology, e.g., U.S. Pat. No. 4,671,958.

### Antisense Therapy

As used herein, “antisense therapy” refers to administration or in situ generation of DNA or RNA oligomers or their derivatives which bind specifically to a target nucleic acid sequence. The binding may be by conventional base pair complementary, or, for example, in the case of binding DNA duplexes, through specific interactions in the major groove of the double helix. By specifically binding to its target DNA or RNA, the function of DNA or RNA is inhibited or suppressed. The antisense oligonucleotides of the present invention may vary in the number of nucleotide residues and may range from about 3 to about 100 nucleotide residues, preferably ranging from about 3 to about 50 nucleotide residues, more preferably from about 3 to about 25 nucleotide residues. In one embodiment the oligonucleotide has less than about 20 nucleotide residues. In another embodiment, the oligonucleotide has about 15 to about 20 nucleotide residues.

Particularly preferred antisense oligonucleotides comprise a nucleic acid sequence which is anticomplementary to the nucleic acid sequence encoding the amino acid sequences of any one SEQ ID NO: 1, SEQ ID NO: 3, or SEQ ID NO: 5 or portions thereof and the like.

Antisense oligonucleotides of the present invention are constructed to prevent the expression of any one of the KLF6 splice variants of the present invention. Antisense oligonucleotides of the invention are nucleotides that bind and prevent or inhibit the transcription and/or translation of the nucleic acid encoding the any one of the KLF6 splice variants of the present invention.

In another embodiment, vectors comprising a sequence encoding an antisense nucleic acid according to the invention may be administered by any known methods, such as the methods for gene therapy available in the art. Exemplary methods are described below. For general reviews of the methods of gene therapy, see, Goldspiel et al., Clinical Pharmacy. 1993; 12: 488-505; Wu and Wu, Biotherapy. 1991; 3: 87-95; Tolstoshev, Ann. Rev. Pharmacol. Toxicol. 1993; 32: 573-596; Mulligan, Science. 1993; 260: 926-932; and Morgan and Anderson, Ann. Rev. Biochem. 1993; 2: 191-217. Methods commonly known in the art of recombinant DNA technology that can be used are described in Ausubel et al., (eds.), Current Protocols in Molecular Biology, John Wiley & Sons New York:N.Y. (1993); Kriegler, Gene Transfer and Expression, A Laboratory Manual, Stockton Press, New York:N.Y. (1990); Dracopoli et al., (eds.) Current Protocols in Human Genetics, John Wiley & Sons, New York:N.Y. (1994).

In one embodiment, a vector is used in which the coding sequences and any other desired sequences are flanked by regions that promote homologous recombination at a desired site in the genome, thus providing for expression of the construct from a nucleic acid molecule that has integrated into the genome (Koller and Smithies, Proc. Natl. Acad. Sci. USA. 1989; 86: 8932-8935; Zijlstra et al., Nature. 1989; 342: 435-438).

Delivery of the vector into a patient may be either direct, in which case the patient is directly exposed to the vector or a delivery complex, or indirect, in which case, cells are first transformed with the vector in vitro, then transplanted into the patient. These two approaches are known, respectively, as in vivo and ex vivo gene therapy.

In a specific embodiment, the vector is directly administered in vivo, where it enters the cells of the organism and mediates expression of the construct. This can be accomplished by any of numerous methods known in the art and discussed above, e.g., by constructing it as part of an appropriate expression vector and administering it so that it becomes intracellular, e.g., by infection using a defective or attenuated retroviral or other viral vector (see, U.S. Pat. No. 4,980,286), or by direct injection of naked DNA, or by use of microparticle bombardment (e.g., a gene gun; Biolistic, Dupont, Wilmington, Del.); or coating with lipids or cell-surface receptors or transfecting agents, encapsulation in biopolymers (e.g., poly-□-1-□4-N-acetylglucosamine polysaccharide; see, U.S. Pat. No. 5,635,493), encapsulation in liposomes, microparticles, or microcapsules; by administering it in linkage to a peptide or other ligand known to enter the nucleus; or by administering it in linkage to a ligand subject to receptor-mediated endocytosis (see, e.g., Wu and Wu, J. Biol. Chem. 1986; 62: 4429-4432).

In another embodiment, a nucleic acid-ligand complex can be formed in which the ligand comprises a fusogenic viral peptide to disrupt endosomes, allowing the nucleic acid to avoid lysosomal degradation, or cationic 12-mer peptides, e.g., derived from antennapedia, that can be used to transfer therapeutic DNA into cells (Mi et al., Mol. Therapy. 2000; 2: 339-47). In yet another embodiment, the nucleic acid can be targeted in vivo for cell specific uptake and expression, by targeting a specific receptor (see, e.g., PCT Publication Nos. WO 92/06180, WO 92/22635, WO 92/20316 and WO 93/14188).

### Interfering RNA

Alternative preferred agents that modulate the activity of any one the KLF6 splice variants of the present invention, are double-stranded RNA species mediating RNA interference (RNAi). RNAi technology, using double stranded RNA (dsRNA) to suppress the expression of any one the KLF6 splice variants of the present invention, or other gene of interest in a homology-dependent manner, can be applied to modulate the activity of any one of the KLF6 splice variants. RNAi is the process of sequence-specific, post-transcriptional gene silencing in animals and plants, initiated by double-stranded RNA (dsRNA) that is homologous in sequence to the silenced gene. Methods relating to the use of RNAi to silence genes in C. elegans, Drosophila, plants, and mammals are known in the art (see, e.g., Fire A, et al., Nature. 1998; 391: 806-811; Fire, A., Trends Genet. 1999; 15: 358-363; Sharp, P. A. Genes Dev. 201; 15: 485-490; Hammond, S. M., et al., Nature Rev. Genet. 2001; 2: 110-1119).

The short interfering RNAs (“siRNA”) to be used in the methods of the present invention are preferably short double stranded nucleic acid duplexes comprising annealed complementary single stranded nucleic acid molecules. In preferred embodiments, the siRNAs are short dsRNAs comprising annealed complementary single strand RNAs. However, the invention also encompasses embodiments in which the siRNAs comprise an annealed RNA:DNA duplex, wherein the sense strand of the duplex is a DNA molecule and the antisense strand of the duplex is a RNA molecule.

In certain preferred embodiments the siRNA comprises nucleic acid sequences that are complementary to the nucleic acid sequence of a portion of the target locus. In certain embodiments, the portion of the target locus to which the RNAi probe is complementary is at least about 15 nucleotides in length. In preferred embodiments, the portion of the target locus to which the RNAi probe is complementary is at least about 19 nucleotides in length. The target locus to which an RNAi probe is complementary may represent a transcribed portion of a polynucleotide encoding any one of the KLF6 splice variants of the present invention, including a portion of any one of SEQ ID NO: 1, SEQ ID NO: 3, or SEQ ID NO: 5. Examples of siRNA within the scope of the present invention include, but are not limited to SEQ ID NO. 20-27.

Another example of siRNA is SEQ ID NO: 60. Additional nucleotides can be added to either the 5′ or 3′ end of the sequence to make it more efficient and/or more stable. Additional nucleotides, for example, can match the KLF6-SV1 sequence.

Delivery of siRNAs can be achieved, for example, by gene therapy, using a viral DNA-based vector to deliver the siRNA to the cells of interest (DNA-directed RNAi-Benitec, Australia), using liposomes (Eurogentec, San Diego, Calif.), or by using chemical modification of siRNA molecules to make them more stable and capable of systemic delivery in the bloodstream (RNA. 2003; 9: 1034-48; RNA. 2004; 10: 766-71).

In a particularly preferred embodiment, the siRNAs of the present invention are super-stable siRNA's which are capable of systemic delivery and which have an increased duration of maximal gene silencing due to increased uptake upon administration and/or increased resistance to nucleases (Nucleic Acids Res. 31:2705-16; 2003). Generation of stable siRNA's can be achieved from several commercial sources, such as Dharmacon RNA Technologies (siSTABLE™-Lafayette, Colo.); Proligo (Boulder, Colo.); Eurogentec (San Diego, Calif.); Sirna Therapeutics (Boulder, Colo.), Promega (Madison, Wis.); Invitrogen (Carlsbad, Calif.); and Intradigm Corporation (Rockville, Md.).

Optimal dosing for the siRNA administration can be determined by a skilled artisan using routine methods.

### Combination Therapy

The present invention also provides combination therapy with the KLF6 splice variant inhibitors and with other modalities used to treat cancers. Such other treatments include chemotherapy (CT) and radiation therapy (RT), including radioimmunotherapy (RIT), and combinations thereof. CT and RT have numerous adverse effects. Patients undergoing CT may develop side effects including nausea, vomiting, diarrhea, hair loss, dry mouth and other oral complications, and cytopenia. In addition to adverse effects of CT and RT, other limiting factors include development of drug resistance by the tumors, and induction of tumor cell growth arrest and senescence. While senescent tumors do not increase in size per se, they still retain the capacity to produce and secrete tumor stimulating mitogens and pro-angiogenic factors that can lead to tumor progression.

Accordingly, tumors continue to be difficult to treat with existing therapies. There is therefore a continued need in the art for non-toxic agents that can effect tumor cell killing and/or potentiate treatment with existing therapies, thereby allowing the use of lower effective doses of the toxic agents. It is one object of the present invention to provide a combination of a KLF6 splice variant inhibitor with CT or RT, or both, to treat cancers in which wild-type KLF6 is decreased or inactivated. In a preferred embodiment, such a combination enables the use of lower doses of either or both CT or RT agents.

The phrase “in combination with” refers to a method of treatment in which two or more treatments are administered collectively or according to a specific sequence, such that they produce a desirable effect.

The term “radiation therapy” or “radiotherapy” refers to use of high-energy radiation to treat cancer. Radiation therapy includes externally administered radiation, e.g., external beam radiation therapy from a linear accelerator, and brachytherapy, in which the source of irradiation is placed close to the surface of the body or within a body cavity. Common radioisotopes used include but are not limited to cesium (¹³⁷Cs), cobalt (⁶⁰Co), iodine (¹³¹I), phosphorus-32 (³²P), gold-198 (¹⁹⁸Au), iridium-192 (¹⁹²Ir), yttrium-90 (⁹⁰Y), and palladium-109 (¹⁰⁹Pd). Radiation is generally measured in Gray units (Gy), where 1 Gy=100 rads.

As used herein, the term “radioimmunotherapy” (RIT) refers to localized delivery of ionizing radiation coupled to a monoclonal antibody (mAb) or other suitable radiation delivery vehicles (i.e., peptides, organic compounds, stem cells, etc.). The radiolabeled mAb is administered in to the blood circulation and localizes to the surface of tumor cells for which the mAb is specific. The present invention encompasses all radioisotopes suitable for use in RIT, including beta-, alpha-, and gamma-emitting isotopes, including but not limited to beta-emitters yttrium-90 (⁹⁰Y), copper-67 (⁶⁷Cu), gamma-emitting iodine-131 (¹³¹I) and Rhenium-186 (¹⁸⁶Re), and alpha-emitters bismuth-213 (²¹³Bi), terbium-149 (¹⁴⁹Tb) and actinium-225 (²²⁵At). Alpha-emitters are well suited for targeting micrometastatic disease and single-tumor cells such as leukemia and other blood-borne disease.

“Chemotherapy” (CT) refers to treatment with anti-cancer drugs. The term encompasses numerous classes of agents including platinum-based drugs, alkylating agents, anti-metabolites, anti-miotic agents, anti-microtubule agents, plant alkaloids, anti-tumor antibiotics, anti-angiogenic agents, kinase inhibitors, proteasome inhibitors, EGFR inhibitors, HER dimerization inhibitors, VEGF inhibitors, antisense molecules, and includes antibodies. Such drugs include but are not limited to adriamycin, melphalan, ara-C, BiCNU, busulfan, CCNU, pentostatin, the platinum-based drugs carboplatin, cisplatin and oxaliplatin, cyclophosphamide, daunorubicin, epirubicin, dacarbazine, 5-fluorouracil (5-FU), fludarabine, hydroxyurea, idarubicin, ifosfamide, methotrexate, altretamine, mithramycin, mitomycin, bleomycin, chlorambucil, mitoxantrone, nitrogen mustard, mercaptopurine, mitozantrone, paclitaxel (Taxol®), vinblastine, vincristine, vindesine, etoposide, gemcitabine, monoclonal antibodies such as Herceptin®, Rituxan®, Campath®, Zevelin® and Bexxar®, irinotecan, leustatin, vinorelbine, STI-571 (Gleevac®), tamoxifen, docetaxel, topotecan, capecetabine (Xeloda®), raltitrexed, streptozocin, tegafur with uracil, temozolomide, thioguanine, thiotepa, podophyllotoxin, filgristim, profimer sodium, letrozole, amifostine, anastrozole, temozolomide, arsenic trioxide, epithalones A and B tretinioin, interleukins (e.g., 2 and 12) and interferons, e.g., alpha and gamma. Antiangiogenic agents include but are not limited to BMS-275291, Dalteparin (Fragmin®) 2-methoxyestradiol (2-ME), thalidomide, CC-5013 (thalidomide analog), maspin, combretastatin A4 phosphate, LY317615, soy isoflavone (genistein; soy protein isolate), AE-941 (Neovastat™; GW786034), anti-VEGF antibody (Bevacizumab; Avastin™), PTK787/ZK 222584, VEGF-trap, ZD6474, EMD 121974, anti-anb3 integrin antibody (Medi-522; Vitaxin™), carboxyamidotriazole (CAI), celecoxib (Celebrex®), halofuginone hydrobromide (Tempostatin™), and Rofecoxib (VIOXX®). Other agents include bortezomib, huBr-E3, Genasense, Ganite, FIT-3 ligand, MLN591RL, MLN27904, MLN576 and MLN518.

The term “chemotherapy” also includes gene therapy with agents such as interferon and the interleukins, i.e., administration of a vector encoding genes for the interferons or interleukins. See e.g., Heller et al., Technol Cancer Res Treat. 2002; 1(3): 205-9.

### Screening for Agonists and Antagonists

Modulatory agents may include agonists and/or antagonists of KLF6 splice variant function which may function by modulating an interaction between KLF6 splice variant polypeptide and wild-type KLF6 or other proteins, or by preventing and/or inhibiting the expression of KLF6 splice variants. Methods for screening for agonists and/or antagonists of KLF6 splice variant function may be achieved using cell-based or cell-free assays.

One can employ any of the well-known two-hybrid assays or other conventional assays to study protein-protein interactions and the disruption thereof by test compounds. In vitro systems can be readily designed to identify lead ligands capable of specifically binding the KLF6 splice variant according to present invention. Generally, such screening assays involve preparation of a reaction mixture, comprising a KLF6 splice variant protein and a test compound, under conditions and for a time sufficient to allow the two compounds to interact (e.g., bind), thereby forming a complex that may be detected. The assays may be conducted in any of a variety of different ways including using microarrays.

Protein binding assays and gel shift assays are useful approaches to detect binding. Exemplary assays include assaying labeled KLF6 splice variant binding to immobilized test peptides, or assaying binding by labeled test peptides to immobilized KLF6 splice variants. Many appropriate assays are amenable to scaled-up, high throughput usage suitable for volume drug screening. Assays may employ a single KLF6 splice variant, multiple variants, fragments of KLF6 splice variants, KLF6 splice variant fusion products.

Detection of KLF6 splice variant interactions may be achieved using specific binding assays such as immunoassays, and biotin/avidin (including streptavidin and neutravidin) binding interactions. Commercial antibodies to VDR and kinases such as cSrc, various labels such as myc and FLAG tags, and the second messenger kinases that can be used to practice the invention are available from several sources, including R&D Systems, Minneapolis, Minn. and Santa Cruz Biotechnology, Santa Cruz, Calif. Antibodies to KLF6 splice variant have been described infra. Such immunoassays include radioimmunoassay, ELISA (enzyme-linked immunosorbant assay), “sandwich” immunoassays, immunoradiometric assays, gel diffusion precipitation reactions, immunodiffusion assays, in situ immunoassays (using colloidal gold, enzyme or radioisotope labels, for example), western blots, precipitation reactions, agglutination assays (e.g., gel agglutination assays, hemagglutination assays), complement fixation assays, immunofluorescence assays, protein A assays, and immunoelectrophoresis assays. In one embodiment, antibody binding is detected by detecting a label on the primary antibody. In another embodiment, the primary antibody is detected by detecting binding of a secondary antibody or reagent to the primary antibody. In a further embodiment, the secondary antibody is labeled. Many means are known in the art for detecting binding in an immunoassay and are within the scope of the present invention.

Other assays, including but not limited to, two-hybrid assays and surface plasmon resonance (SPR) assays, could be used to identify such interactions. Yeast two-hybrid assays as described by Young and Ozenberger in U.S. Pat. No. 5,989,808, which is herein incorporated by reference. SPR is described in Wegner et al., (2002) Anal. Chem. 74: 5161-5168; Cooper et al., Anal Bioanal Chem. 2003; 377(5): 834-42; and Gambari, Curr Med Chem Anti-Canc Agents. 2001; 1(3): 277-91).

### Microarrays

The terms “array” and “microarray” are used interchangeably and refer generally to any ordered arrangement (e.g., on a surface or substrate) or different molecules, referred to herein as “probes”. Each different probe of an array specifically recognizes and/or binds to a particular molecule, which is referred to herein as its “target”. Microarrays are therefore useful for simultaneously detecting the presence or absence of a plurality of different target molecules, e.g., in a sample. The presence or absence of that probe's target molecule in a sample may therefore be readily determined by simply analyzing whether a target has bound to that particular location on the surface or substrate.

Conventional mircroarrays generally comprise a solid non-porous substrate, such as glass slide or a computer chip. In a typical microarray application, the substrate is contacted with a sample containing biomaterials to be analyzed. The substrate is then contacted with probe molecules such as labeled nucleic acids or polypeptides or other molecules. The labeled molecules bind with the molecules in the sample. The unbound probe molecules are removed, for example, by washing, and the microarray is then read by a suitable signal detection device, for example, by fluorescence emission.

For example, one embodiment comprises anchoring a protein, e.g., KLF6 splice variant, or a test ligand, onto a solid phase and detecting complexes of the protein and the test ligand that are on the solid phase at the end of the reaction and after removing (e.g., by washing) unbound ligands. For example, in one preferred embodiment of such a method, a protein may be anchored onto a solid surface and a labeled compound or polypeptide is contacted to the surface. After incubating the test ligand for a sufficient time and under sufficient conditions that a complex may form between the protein and the test compound or polypeptide, unbound molecules of the test ligand are removed from the surface (e.g., by washing) and labeled molecules which remain are detected.

In another, alternative embodiment, molecules of one or more different test compounds are attached to the solid phase and molecules of a protein (for example, a labeled KLF6 splice variant polypeptide) may be contacted thereto. In such an embodiment, the molecules of different test compounds are preferably attached to the solid phase at a particular location on the solid phase so that test compounds that bind to a protein may be identified by determining the location of bound proteins on the solid phase or surface. Again, mutant and variant proteins may be used as test compounds.

In addition, screening can be done using wild-type KLF6 as an antagonist to a well containing an KLF6 splice variant and a test compound, to which a test compound is added to the well in the presence and absence of wild-type KLF6. Any binding and formation of a test compound/KLF6 splice variant interaction should be inhibited by addition of unlabeled wild-type KLF6 which binds to KLF6 splice variant.

Automated multiwell formats are the best developed high-throughput screening systems. Automated 96-well plate-based screening systems are the most widely used. The current trend in plate based screening systems is to reduce the volume of the reaction wells further, thereby increasing the density of the wells per plate (96-well to 384-, and 1536-well per plate). The reduction in reaction volumes results in increased throughput, dramatically decreased bioreagent costs, and a decrease in the number of plates which need to be managed by automation. For a description of protein arrays that can be used for high-throughput screening, see U.S. Pat. Nos. 6,475,809, 6,406,921, and 6,197,559, herein incorporated by reference.

### KLF6 Splice Variant Activity Assays

Once a candidate compound or compounds that specifically bind to a KLF6 splice variant protein has been designed or identified and characterized, it can be used in assays to determine if it modulates the activity of KLF6 splice variant. Examples of assays are described in the below and in the Examples.

Activity assays are generally designed to measure the activity of a target protein in the presence or absence of a test agent. Activity assays, including but not limited to mammalian transfection assays in which KLF6 splice variant activity is observed, could be used to identify lead ligands. For example, ligands could be screened for their ability to modulate the interaction KLF6 splice variant with wild-type KLF6.

One method used for screening for a ligand that modulates the activity of a KLF6 splice variant comprises the steps of (a) contacting a test compound with a KLF6 splice variant polypeptide; and (b) determining whether said test compound specifically binds said polypeptide. In addition, the method can comprise the steps of (a) adding a test compound to a cell comprising the KLF6 splice variant and the wild-type KLF6; and (b) comparing the KLF6 splice variant activity before and after the addition of the compound. Further approaches to this method involve adding a test compound to a control sample comprising a cell which lacks KLF6 splice variant activity.

There are numerous approaches for identifying compounds that affect genomic activity of nuclear proteins. In one embodiment, this comprises the steps of (a) adding a test compound to a cell comprising a KLF6 splice variant and a wild-type KLF6; and (b) comparing the genomic activity before and after. A selective genomic activity can be measured by conventional means. Preferably, the increase or positive effect of genomic activity, as measured, is a two-fold alteration in, e.g. transcription of cell cycle inhibitory genes such as p21, cell adhesion genes such as E-cadherin, or pro-angiogenic genes such as VEGF, after addition of said test compound to a cell in the presence of KLF6 splice variant and wild-type KLF6 when compared to genomic activity with test compound in the absence of KLF6 splice variant, and wherein no change is observed in genomic activity after addition of said test compound.

Assays to detect changes, e.g., increases or decreases in transcription of a gene into e.g., mRNA are well-known in the art. Such assays include RT-PCR, including quantitative RT-PCR, Northern hybridization, transfection assays of the gene-of-interest linked to a reporter gene, gene-expression arrays, etc.

The present invention is further described by way of the following particular examples. However, the use of such examples is illustrative only and is not intended to limit the scope or meaning of this invention or of any exemplified term. Nor is the invention limited to any particular preferred embodiment(s) described herein. Indeed, many modifications and variations of the invention will be apparent to those skilled in the art upon reading this specification, and such “equivalents” can be made without departing from the invention in spirit or scope. The invention is therefore limited only by the terms of the appended claims, along with the full scope of equivalents to which the claims are entitled.

### EXAMPLES

The following Examples are provided to illustrate the invention without being limiting in any way.

### Example 1

### Detection of KLF6 Splice Variants in Transfected Cells

This example describes the detection of splice variants of the KLF6 gene, produced from a common polymorphism, IVSΔA.

### Materials and Methods

Sequence Analysis. All sequencing was performed either on an ABI Prism 3730 or 3700 automated DNA analyzer and sequence data was analyzed using the Sequencher 4.1 program (Gene Codes Corporation) or Phred/Phrap/Consed (University of Washington). Genomic DNA was amplified to generate a 171 bp amplicon using the following wtKLF6 specific primers: ΔATG Fwd: 5′-CGG GCA GCA ATG TTA TCT GTC CTT C-3′ (SEQ ID NO: 7) and ΔATG Rev: 5′-TTC TGA GGC TGA AAC ATA GCA GGG-3′ (SEQ ID NO: 8). The PCR product was then either sequenced per standard protocols or digested with BsaAI (New England Biolabs) using the manufacturer's recommendations and resultant products were gel electrophoresed on a 1.5% TAE gel for 1 hour at 80V and then visualized by ethidium bromide staining.

wtKLF6 minigene constructs. To amplify the 6.2 kb KLF6 genomic locus including approximately 100 bp of the 5′ UTR and 300 bp of 3′ UTR, 200 ng of human genomic DNA was amplified using the following set of primers wtKLF6-1F: 5′-TTG CAG TCA GTC CGG TGT TTG-3′ (SEQ ID NO: 9) and wtKLF6-4R1: 5′-GGT GCT ATG CCG CTT CTT ACA GGA C-3′ (SEQ ID NO: 10) using the EXPAND Long Template PCR system (Roche). PCR was performed according to manufacturer's suggestions. The resultant 6.2 kb PCR product was purified (Qiagen) and then subcloned into the TOPO TA expression vector (Invitrogen). All intron/exon boundaries and 5′ UTR and 3′ UTR regions of the construct used in this study were sequenced in both orientations prior to use.

Cell culture and transient transfection. 293T cells were obtained from the American Tissue Culture Collection (ATCC). All transient transfection was performed with Lipofectamine 2000 reagent according to the manufacturer's protocol (Life Technologies). Stable cell lines were generated by cotransfection of the minigene expression construct with a puromycin expressing plasmid. Transfected cells were selected with 2 μg/ml of puromycin.

### Results and Discussion

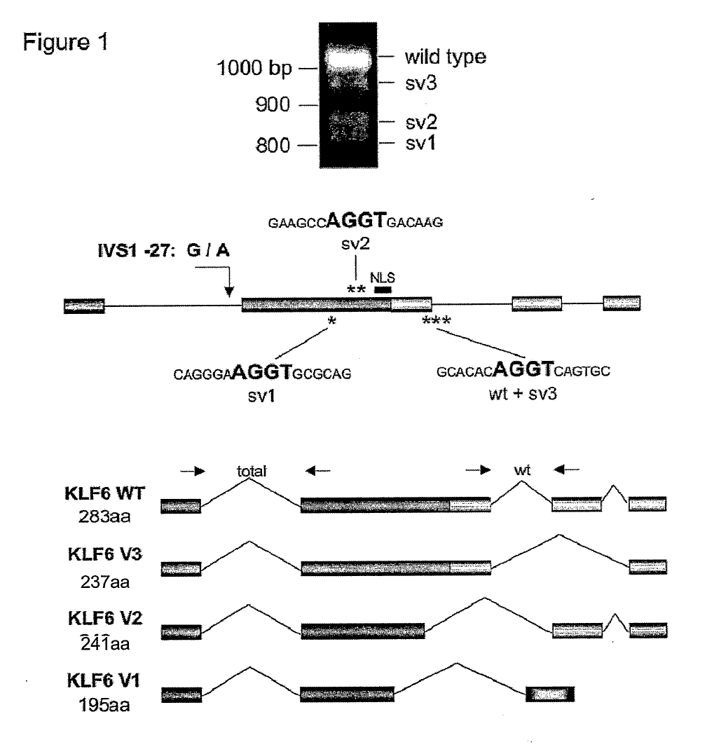

A potential biologic role for the IVSΔA allele was examined in cultured cells by expressing full-length 6.2 kb KLF6 minigene constructs, containing the wtKLF6 sequence. Findings stemming from this experiment suggested that the wtKLF6 gene is alternatively spliced. Direct cDNA sequence analysis from 293T cells transfected with the minigene constructs confirmed the presence of three alternatively spliced KLF6 gene transcripts, KLF6sv1, sv2 and sv3 (SEQ ID NOS: 1-6). These alternatively spliced products, arising from the use of native cryptic splice sites within exon 2 (FIG. 1), predicted protein isoforms lacking either parts or all of the wtKLF6 activation and/or DNA binding domain. KLF6sv1 and KLF6_{SV3} contain novel 21 and 12 amino acid carboxy domains, respectively, resulting from out-of-frame splicing of their terminal exons. Additionally, a known base pair variation exists in wtKLF6 which can be incorporated into sv2 and sv3 (SEQ ID NOS: 50-51).

### Example 2

### KLF6 Splicing in Human Cancers

This example describes the detection of KLF6 splice variants in prostate, ovarian and hepatocellular cancer, as well as in benign, and metastatic cells.

### Materials and Methods

Sequence and Genotype Analysis. Genomic DNA was extracted from peripheral blood samples as previously described (Carpten et al., Nat Genet. 2002; 30: 181-4; Wang et al., Cancer Res. 2001; 61: 6494-9; Stanford et al., Cancer Epidemiol Biomarkers Prev. 1999; 8: 881-6). DNA from 142 probands from the JHU Familial Prostate Cancer Registry was analyzed by direct sequence analysis of the second exon and intron/exon boundaries using wtKLF6 specific primer combinations as previously described (Narla et al., Science. 2001; 294: 2563-6). PCR products were directly sequenced in both orientations after purification (Qiagen, QIAquick PCR purification kit). All sequencing was performed as indicated in Example 1. In addition, all JHU genotypes were reconfirmed at a second institutional site by BsaAI restriction enzyme digest. Genotypes for the Mayo Clinic samples were reconfirmed by pyrosequencing, as previously described (Wang et al., Cancer Res. 2001; 61: 6494-9), using the same wtKLF6 primers described above except that one primer was biotinylated. As a control, more than half of all sample results were independently confirmed by a second institution. To evaluate the possibility of combining the data sets, logistic regression with an interaction term (institution by KLF6 carrier status) was performed to determine if the OR's were significantly different between the registries (Slager and Schaid, Am J Hum Genet. 2001; 68: 1457-1462; Weir, Genetic Data Analysis 11. Methods for Discrete Population Genetic Data. Sinaur Assoc., Inc. Mass (1996); Sasieni, P. Biometrics. 1997; 53: 1253-61). This analysis was conducted for the entire group, as well as all the subgroups. No interaction was statistically significant and thus the data sets were also analyzed as a combined set.

Ovarian tumor (EOC) preparation. Tumor specimens were collected and analyzed under IRB approval. Histologic diagnosis was validated by pathology review at the University of Iowa Institutional Gynecological Oncology Tumor Board. Tumors where staged in accordance with the International Federation of Gynecology and Obstetrics (FIGO) surgical staging system. Tumor samples were snap-frozen at the time of surgery in liquid nitrogen. DNA isolation and preparation techniques have been reported previously (Skilling et al., Oncogene. 1996; 13: 117-23). Ten additional samples were collected and analyzed with consent of the institutional review board from Regional Medical Center in San Jose, Calif. Normal DNA was obtained from microdissected normal tissue in the region of the tumor or from margins with no evidence of cancer. In all cases, a 5-μm section stained with H&E was used as a histologic reference for normal and tumor-derived tissue. Manual microdissection was performed on serial 20-μm sections, and DNA was subsequently extracted using commercial reagents (Ambion, Austin, Tex.). Normal whole ovarian tissue cDNA was commercially obtained (Clontech).

IVSΔA minigene constructs: In addition to the wtKLF6 construct described in Example 1, the IVSΔA mutation was introduced into the full length minigene construct by site-directed mutagenesis according to the manufacturer's protocol (Quick-Change, Stratagene). The primers used for mutagenesis were as follows: IVSΔA-F: 5′-GTC ATG GCA ATC ACA TGC CTT CTC TGG TT-3′ (SEQ ID NO: 11) and IVSΔA-R: 5′-AAC CAG AGA AGG CAT GTG ATT GCC ATG AC-3′ (SEQ ID NO: 12).

Cell culture and transient transfection. Cell culture was performed according to the method set forth in Example 1. BPH1 and PC3M cells were obtained from the American Tissue Culture Collection (ATCC).

PCR Analysis. Prostate tissue derived cDNAs were amplified with the primers described in Example 1. PCR was performed according to manufacturer's suggestions (Applied Biosystems). The resultant PCR products were separated on 1.5% agarose gel by electrophoresis. In order to confirm the sequence of each of the KLF6 splice variants, the PCR products were gel purified and subcloned into the TOPO TA vector (Invitrogen); five independent clones for each PCR product were sequenced.

RNA and qRT-PCR analysis. All RNA samples were collected and extracted as previously described (Brummelkamp et al., Science. 2002; 296:550-3. Cell line RNA was extracted using the Rneasy Mini kit (Qiagen). All RNA was treated with DNAse (Qiagen). A total of 1 ug of RNA was reverse transcribed per reaction using first strand complementary DNA synthesis with random primers (Promega). qRT-PCR was performed using the following PCR primers on an ABI PRISM 7900HT Sequence Detection System (Applied Biosystems): wtKLF6 Forward: 5′-CGG ACG CAC ACA GGA GAA AA-3′ (SEQ ID NO: 13) and Reverse: 5′-CGG TGT GCT TTC GGA AGT G-3′ (SEQ ID NO: 14). TotalKLF6 Forward: 5′-CTG CCG TCT CTG GAG GAG T-3′ (SEQ ID NO: 15) and Reverse: 5′-TCC ACA GAT CTT CCT GGC TGT C-3′ (SEQ ID NO: 16). GAPDH Forward: 5′-CAA TGA CCC CTT CAT TGA CC-3′ (SEQ ID NO: 17) and GAPDH Reverse: 5′-GAT CTC GCT CCT GGA AGA TG-3′ (SEQ ID NO: 18). All experiments were done in triplicate and normalized to GAPDH. In order to calculate the fold change in wtKLF6 alternative splicing, the fold change in total KLF6 (wtKLF6+alternatively spliced KLF6 transcripts) was divided by the fold change in wtKLF6 alone.

Western Blot analysis. BPH1, PC3M or 293T cells grown in 12-well dishes were transfected with 0.5 μg per well of the minigene expression vectors. Cell extracts for western blotting were harvested in RIPA buffer 24 h following transfection (Santa Cruz Biotechnology standard protocol). Equal amounts of protein (50 μg) as determined by the BioRad DC Protein quantification assay were loaded and separated by polyacrylamide gel electrophoresis and transferred to nitrocellulose membranes. Western blotting was performed using monoclonal antibodies to KLF6 and a goat polyclonal antibody to actin (SC-1615).

Densitometric Analysis. Enhanced chemiluminescent images of immunoblots were analyzed by scanning densitometry and quantified with a BIOQUANT NOVA imaging system. All values were normalized to actin and expressed as fold changes relative to control.

Generation of KLF6 monoclonal antibodies. A 67-kDa GST-fusion peptide containing amino acids 28-199 of the human KLF6 activation domain pGEX-2-P* and the following peptide: EKSLTDAHGKGVSGVLQEVMS (SEQ ID NO: 19) were purified and used to generate the 2A2 and 9A2 monoclonal KLF6 antibodies respectively. (The KLF6 monoclonal antibody (2A2) is capable of recognizing all KLF6 isoforms; the 9A2 antibody is specific for KLF6_{SV1}).

Microarray methods and analysis. Complementary DNA microarray analysis of gene expression was done essentially as described (available at http://www.microarrays.org). Based on the latest build of Unigene, our 10K human cDNA microarray covers about 5,520 known, named genes and 4,464 ESTs. Primary analysis was done with the Genepix software package. Cy3-to-Cy5 ratios are determined for the individual genes along with various other quality-control parameters (for example, intensity over local background). Furthermore, bad spots or areas of the array with obvious defects were manually tagged. Flagged spots were not included in subsequent analyses. These values were then imported into a Microsoft Access database. Before clustering, the normalized median of ratio values of the genes were log2 transformed, filtered for presence across arrays, and selected for expression levels and patterns depending on the experimental set. Average-linkage hierarchical clustering of an uncentered Pearson correlation similarity matrix was applied with the program Cluster11, and the results were analyzed; figures were generated with the program TreeView.

### Results and Discussion

KLF6 splice variants in prostate cancer. To assess potential differences in in vivo expression patterns, RNA was isolated from 6 normal prostate tissue samples and 15 prostate tumors and the amount of wtKLF6 and KLF6 splice variants was determined by qtRT-PCR. Prostate tumors had significantly higher splicing ratios (x=0.83) and a higher total/wtKLF6 ratio, than normal prostate tissue (x=0.41) (p<0.01). Restated, normal prostate expressed twice the relative amount of wtKLF6 tumor suppressor message to splice products when compared to tumors (p<0.01). Real time PCR also demonstrated that 50% of KLF6 mRNA is KLF6_{SV1}, which is comparable to many cancer cell lines (e.g., PC3 cells, HepG2 cells).

The effect of the IVSΔA variant on KLF6 splicing was analyzed in noncancerous and cancerous prostatic tissues. Tissue from seven men with benign prostatic hypertrophy, a non-cancerous condition, was studied. Four samples were homozygous for the wtKLF6 allele, 3 were heterozygous. The IVSΔA allele was again associated with an approximately 40% increase in KLF6 splicing (p<0.01).

### Results

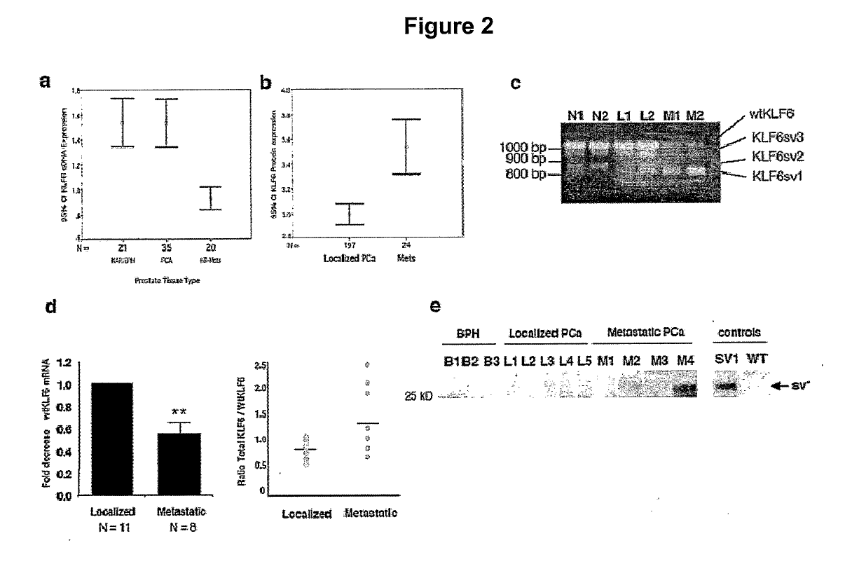

KLF6 gene expression profiles from benign prostate tissue, clinically localized prostate cancer, and metastatic prostate tumors (Dhanasekaran S M, et al. Nature. 2001; 412: 822-6) were compared using a 9,984-element (10K) human cDNA microarray. Using a significance analysis of microarray (SAM) test, a number of genes were shown to be differentially regulated in localized vs. metastatic disease. KLF6 cDNA expression was shown to be downregulated 1.64 fold in metastatic tissue as compared to localized tumor and non malignant BPH tissue samples (one way ANOVA p<0.0001) (FIG. 2a) consistent with its role as a tumor suppressor gene. In light of the decreased KLF6 expression pattern seen in metastatic tissues, prostate cancer samples from 197 clinically localized tumors and 24 hormone-refractory prostate tumors were evaluated by immunohistochemistry for KLF6 protein expression. An increase in cytoplasmic KLF6 staining was observed in metastatic, hormone-refractory prostate tumors as compared to clinically localized disease with a mean staining intensity of 3.0 and 3.54, respectively (p<0.001; FIG. 2b).

In addition, a strong link was found between IVSΔA and PCa recurrence. Briefly, in a blinded study, 58 DNA samples obtained from a well-annotated set of tumors were genotyped for the presence of the IVSΔA SNP and then compared these results to long-term clinical characteristics of the patients. The presence of the IVSΔA SNP was associated with an almost 4× greater risk of recurrence (OR 3.9; 95% CI 3.0-4.8; p<0.01) and 5.5× greater risk of metastatic disease (95% CI 4.3-6.7; p<0.01). This is the first genetic SNP linked to the risk of prostate cancer recurrence or metastasis.

Similar results were obtained in tumor sections from epithelial ovarian cancer, in which all positive staining was localized to the cytoplasm with no detectable nuclear staining (data not shown). Moreover, staining intensities were positively correlated (p<0.001).

In order to potentially explain the difference in KLF6 mRNA and protein expression in metastatic prostate cancer and the findings identifying the cytoplasmic accumulation of the KLF6_{SV1} and KLF6_{SV2} proteins, RT-PCR of cDNA derived from normal and cancerous prostate tissue was performed. Consistent with the immunohistochemical data, a marked relative overexpression of the KLF6 splice variants, as compared to wtKLF6, was seen in metastatic tissues (FIG. 2c). Real time PCR analysis of the localized and metastatic prostate cancer cDNA confirmed that whereas wild type KLF6 levels were significantly decreased in metastatic tumors, a subset of these tumors ( 3/7) had significantly increased levels of KLF6 alternative splicing (FIG. 2d). Protein extracts from normal prostate tissue, localized prostate cancer, and metastatic disease were then immunoblotted using the KLF6 monoclonal antibody (2A2). Overexpression of the KLF6_{SV1} protein was seen only in metastatic samples ( 2/4) analyzed (FIG. 2e). No detectable KLF6_{SV1} protein was seen in either benign prostatic hyperplasia (BPH) ( 0/3) or localized prostate cancer samples ( 0/5). Collectively these findings suggest that the ratio of wild type to splice form KLF6 mRNA is consistently decreased in metastatic prostate cancer, through a decrease in wild type KLF6 expression, an increase in splicing, or both.

A KLF6_{SV1} specific monoclonal antibody (9A2) was generated to examine KLF6_{SV1} splice variant expression in the original localized prostate cancer samples and hormone-refractory prostate tumors analyzed. The KLF6_{SV1} protein is predicted to contain a novel 21 amino acid carboxy domain resulting from out-of-frame splicing of exon 3. This 21 amino acid sequence target was used to generate a KLF6_{SV1} specific monoclonal antibody. The specificity of this antibody was evaluated by western blotting of protein extracts expressing either wtKLF6, KLF6_{SV1}, or KLF6_{SV2} (data not shown).

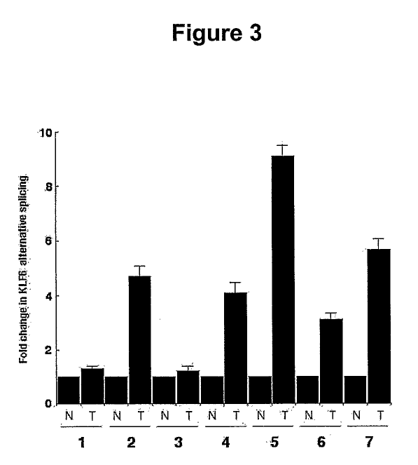

KLF6 splicing in Hepatocellular Carcinoma. The detailed analysis of KLF6 alternative splicing in prostate has been extended to studies of hepatocellular carcinoma. Matched tumor and surrounding normal livers from 7 hepatic explants with HCC were analyzed, and a consistent increase in KLF6 splicing was detected in all tumors compared to their matched normal liver (p<0.005) (FIG. 3).

KLF6 Splicing in Ovarian Cancer. Based on the previous discovery of alternative splice forms of KLF6 in prostate cancer, the presence of the two variants, KLF6_{SV1} and KLF6_{SV2} in the EOC tumors was examined. RNA expression levels from 41 fresh frozen patient samples were analyzed by RT-PCR. The ratio between total (wild type and splice variants)/wild type KLF6 is calculated and represented as a numerical value in the “splicing index”. The splicing index from 3 patient samples was calculated to be 1.9, 0.6, and 0.1, respectively (data not shown). Direct sequencing of the amplified transcripts was performed to determine which splice form was being made in these tumors. From different patient samples it was confirmed that the alternatively spliced KLF6 message corresponds to KLF6_{SV1}.

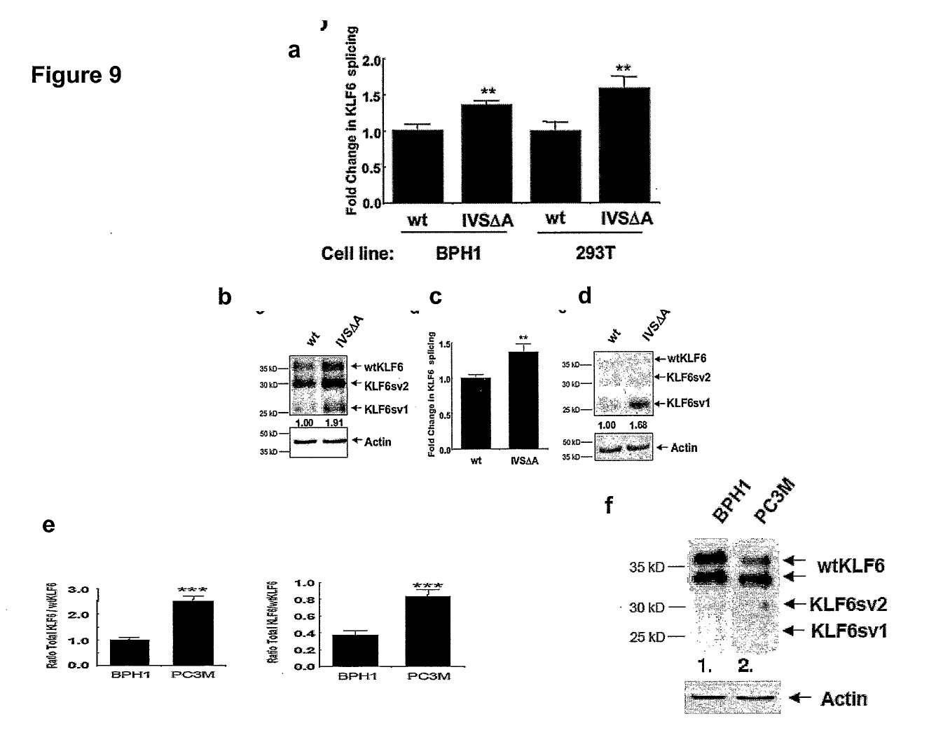

KLF6 Splicing in Prostate Cancer Cell Lines. The IVSΔA and wtKLF6 minigene constructs were transiently and stably transfected into a range of cell types and the KLF6-related mRNAs and proteins were compared by qtRT-PCR and western blotting, respectively. Regardless of cell line used, the IVSΔA allele resulted in increased alternative splicing by 30-50% in BPH1 and 293T cells (FIGS. 9a,c) (p<0.001). The levels of wtKLF6 expression in IVSΔA expressing cells were similar to GTG expressing cells suggesting that the changes in the splicing ratio were secondary to increased alternative splicing and not decreased levels of wtKLF6. The presence of the IVSΔA allele resulted in increased levels of both variant proteins. The ratio of splice variants to wtKLF6 protein was quantitated by densitometry and shown to be increased by approximately 90% (FIG. 9b). Consistent with the RNA data, KLF6_{SV1} and KLF6_{SV2} protein was also increased approximately 60% as compared to the wtKLF6 and minigene control (FIG. 9d).

Increased Alternative Splicing of KLF6 in Metastatic Cancer Cell Lines. The KLF6 minigene construct was transiently transfected into two prostate cell lines, the benign prostatic hyperplasia cell line BPH1 and the metastatic prostate cancer cell line PC3M.

The levels of wtKLF6 and alternative splicing in both of these cell lines was determined and compared at both mRNA and protein levels by qtRT-PCR and western blotting, respectively. A marked reduction in wtKLF6 mRNA levels and increased production of alternatively spliced KLF6 transcripts was seen both with the transfected minigene and at the endogenous level in the metastatic (PC3M) compared to the non-metastatic (BPH1) derived cell line (FIG. 9e). [PC3M cells had a significantly higher splicing ratio (x=2.4), than the BPH1 cell line (x=1.0) (***p<0.0001). Furthermore, the PC3M cell line had a significantly higher endogenous splicing ratio (x=0.83) than did the BPH1 cell line (x=0.41) (***p<0.0001)]. In accord with these findings, western blot analysis revealed a significant decrease in wtKLF6 protein levels and an increase in KLF6_{SV1} and KLF6_{SV2} levels which resulted in a 2.8 fold increase in the ratio of alternatively spliced KLF6 to wtKLF6 in the PC3M cells (FIG. 9f).

### Example 3

### Comparison to wtKLF6 and Correlation as a Method of Diagnosis/Prognosis

This example describes the relationship between levels of wtKLF6 and KLF6 splice variants. A ratio of these two factors is present in tissues throughout tumor progression. Therefore, this ratio is useful as both a diagnosis of tumor status, as well as a method for determination of disease progression.

### Materials and Methods

RNA and qRT-PCR analysis. See Example 2 for relevant methodology.
Sequence and Genotype Analysis. See Examples 1 and 2 for relevant methodology.

### Results and Discussion

### Correlation of KLF6 Splicing and Tumor Grade

Prostate tumor samples were haplotyped and the splicing ratios determined for all nine of the 15 originally analyzed samples for which Gleason scores were available The IVSΔA allele was associated with a 30% increase in splice ratio in tumors graded 3+3 (* p<0.01) (G/G n=3; G/A n=2) and a 40% increase in the higher grade 4+4 (** p<0.001) (G/G n=2; G/A n=2). Taken together, the cell culture and tissue results suggested that the IVSΔA allele results in increased splicing, while an association between increased KLF6 splicing and prostate cancer was noted.

In addition, ovarian tumors (EOC) were also examined to determine if a similar correlation existed between the expression of KLF6 splice variants and EOC clinical-pathologic factors including tumor grade, histology and FIGO stage. Results suggest that increased KLF6 isoform expression, primarily KLF6_{SV1}, is associated with a more aggressive tumors and may be specific to the histological tumor type. A KLF6 monoclonal antibody was used to detect both wt and splice KLF6 staining intensity and localization in 41 ovarian tumors.

Splicing ratios and staining intensities were positively correlated (p<0.001). In addition, to determine relative contribution of staining by each isoform, the RNA expression levels of KLF6_{SV1} and −KLF6_{SV2} were compared. There was an approximate 2-fold increase in KLF6_{SV1} mRNA expression in tumors with high cytoplasmic staining (p<0.004). Compared to KLF6_{SV1}, KLF6_{SV2} was expressed at low levels in 75% of samples and remained unchanged regardless of staining intensity. As indicated above, all staining was cytoplasmic.

Finally, a 2-fold increase in KLF6 splicing index in poorly differentiated grade III tumors was observed compared to well to moderately differentiated grade I or II tumors (p<0.035). Similarly, 18/19 tumors with high KLF6 staining patterns were grade III tumors. Since only 4/12 tumors were grade II, low staining pattern analysis was not informative. Nonetheless, this staining/grade distribution pattern was statistically significant (p<0.05). Additionally, a 30% increase in KLF6 alternative splicing was detected in stage III/IV tumors when compared to stage I/II but due to the low number of stage I/II tumors statistical significance was not achieved. Differences in splicing were also evident when comparing serous tumors to non-serous; the serous tumors had a 50% increase in splicing (p<0.05).

### Example 4

### Cellular Localization of KLF6 Splice Variants

This example describes the cellular distribution of KLF6 and the KLF6SVs. Localization may implicate a role for splice variants in alternate cell signaling pathways which result in the inactivation of wtKLF6 and other binding proteins.

### Materials and Methods

KLF6 FLAG constructs. wtKLF6 cDNA was cloned into EcoRI and XbaI sites of pCI-neo5. KLF6sv1 and sv2 expression vectors were generated by subcloning the appropriate full length cDNA into the EcoRI site of the pCI-neo expression vector. wtKLF6, KLF6_{SV1}, and KLF6_{SV2} constructs were generated by cloning the appropriate cDNA into the EcoRI site of the FLAG expression vector pcDNA3 (Invitrogen).

Cell culture and transient transfection. Cell culture was performed according to the method set forth in Example 1. HEK293 cells were obtained from the American Tissue Culture Collection (ATCC).

Immunocytochemistry. 293HEK cells were cultured on growth promoting cover slips (Fisherbrand) in 12 well dishes. At 24 h cells were transfected with pcDNA3 FLAG (empty vector), FLAG-wtKLF6, FLAG-sv1, FLAG-sv2. At 18 h, cells were washed twice with ice cold saline, fixed for 10 min with ice cold methanol, and then ice cold acetone for 1 minute. Blocking was with 5% BSA/PBS for 10 min prior to incubation with primary FLAG monoclonal antibody (1:100) and subsequent detection with Texas Red conjugated anti-mouse secondary antibody (Vector laboratories). Additionally, blotting utilizing a rabbit polyclonal antibody recognizing all forms of KLF6 was utilized (R-173 Santa Cruz Biotechnology)

EOC tumor immunocytochemistry. EOC tumor sections were prepared as described above. Sections (5 μm) were deparaffinized and rehydrated through serial xylene followed by ethanol dilutions. Endogenous peroxidase was blocked by incubating sections in 3% H₂O₂ for 10 min. The sections were then washed in tap water for 5 min. Antigen retrieval was done by immersing slides in boiling citric acid buffer (10 mM, pH 6.0) for 15 min. Slides were cooled for 20 min at room temperature and then washed in tap water for 5 min and in 1×PBS (pH 7.4) 2×5 min. Slides were blocked for 10 min in 10% goat serum. Primary KLF6 (Zf-9) antibody (Santa Cruz) diluted 1:100 in 1×PBS+0.25% BSA was incubated for 1.5-2 hours. The slides were then washed 2×5 min in 1×PBS (pH 7.4) and incubated in polyclonal-Hrp anti-rabbit IgG for 30 min. Following the incubation with the secondary antibody, slides were washed 2×5 min in 1×PBS, incubated in DAB/peroxide solution for 5-8 min, rinsed in tap water and counterstained with hematoxylin. The slides were then dehydrated and mounted. Human tonsil and normal ovarian tissue samples were used as positive controls. Negative controls were used in all runs by replacing the primary antibody with blocking buffer. Reactivity was measured using a semiquantitative scale for intensity of 0 (negative), 1 (weak), 2 (moderate), and 3 (strong). Stain distribution was also quantified: 0 (0%), 0.5 (5%), 0.75 (10%), 1 (15%), 1.5 (30%), 2 (55%), 2.5 (65%), 2.75 (75%) and 3 (90%) of the slide. An intensity score [(intensity+1)+(% positive cells)] was determined to combine these two parameters and the values plotted and assigned a designation of positive vs. negative based on score.

### Results and Discussion

Immunocytochemistry of 293HEK cells (human embryonal fibroblast) expressing either FLAG-wtKLF6, FLAG-KLF6sv1, or FLAG-KLF6sv2 was performed using both the polyclonal KLF6 antibody which recognizes all three proteins, and a FLAG monoclonal antibody. Whereas wtKLF6 localized primarily to the cell nucleus, both the KLF6_{SV1} and KLF6_{SV2} proteins accumulated predominantly in the cytoplasm without evidence of nuclear localization. This compartmentalization reflects the absence of the nuclear localization signal (NLS) in these variants, which maps to the terminal portion of the activation domain and the DNA binding domain (Quadrini and Bieker, J. Biol. Chem. 2002; 277: 32243-52). This region is preserved only in wtKLF6 but not in the KLF6 splice variants SV1 and SV2.

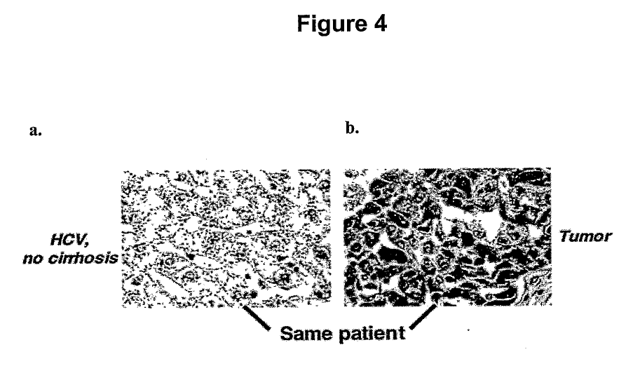

Correlating with increased splicing in cell culture, similar localization is noted in tissue samples as well. In tissue sections of HCC, a consistent increase in cytoplasmic accumulation of KLF6 is noted compared to normal tissue from the same patient (FIG. 4a-b). This finding is similar to what has been seen in colon cancer and the pattern of splice form expression in cultured cells. In addition, in the EOC (ovarian) tumor samples, all positive staining was localized to the cytoplasm with no detectable nuclear staining (data not shown) Moreover, splicing ratios and staining intensities were positively correlated (p<0.001) (data not shown). Thus, based on these cell culture studies, the increased staining in tumors reflects accumulation of splice forms that lack a nuclear localization signal.

### Example 5

### Inhibition of KLF6 Splice Variants Using RNA Interference

This example describes a method to block splice variant function via use of RNA Interference (RNAi). Splice variants serve as dominant negative regulators of wtKLF6 function. Inhibition of splice variants with siRNA serves to remove this inhibition, and restore wtKLF6 anti-tumor effects.

### Materials and Methods

Cell culture and transient transfection. Cell culture was performed according to the method set forth in Examples 1 and 2. Additionally SKOV3 and HeLa cells were obtained from the American Tissue Culture Collection (ATCC).

Western Blot analysis. Protocols followed those set forth in Example 2.

RNA and qRT-PCR analysis. Protocols followed those set forth in Example 2.

pSUPER plasmid construction and transfection. The pSUPER-si-sv1 and pSUPER-si-sv2 plasmids used to downregulate KLF6_{SV1} and KLF6_{SV2} expression were constructed similarly as described using this pSUPER vector 27 (generously provided by R. Agami). To insert the targeting sequence, DNA oligos were designed and cloned into the BglII-HindIII sites of the pSuper vector:

Si-sv1-F:

(SEQ ID NO: 20)

GATCCCCAGGCTTTTCTCCTTCCCTGGCttcaagagaGCCAGGGAAGGAG
AAAAGCCTTTTGGAAA
Si-sv1-R:

(SEQ ID NO: 21)

AGCTTTTCCAAAAAGGCTTTTCTCCTTCCCTGGCtctcttgaaGCCAGGG
AAGGAGAAAAGCCTGGG
Si-sv2-F:

(SEQ ID NO: 22)

GATCCCCGCCAGGAGAAAAGCCTTACttcaagagaGTAAGGCTTTTCTCC
TGGCTTTTTGGAAA
Si-sv2-R:

(SEQ ID NO: 23)

AGCTTTTCCAAAAAGCCAGGAGAAAAGCCTTACtctcttgaaGTAAGGCT
TTTCTCCTGGCGGG
Luc-F:

(SEQ ID NO: 24)

GATCCCCTACTTCGAAATGTCCGTTCttcaagagaGAACGGACATTTCGA
AGTATTTTTGGAAA
Luc-R:

(SEQ ID NO: 25)

AGCTTTTCCAAAAATACTTCGAAATGTCCGTTCtCtCttgaaGAACGGAC
ATTTCGAAGTAGGG
Si-wtKLF6-F:

(SEQ ID NO: 26)

GATCCCCTGGCGATGCCTCCCCCGACttcaagagaGTCGGGGGAGGCATC
GCCATTTTTGGAAA
Si-wtKLF6-R:

(SEQ ID NO: 27)

AGCTTTTCCAAAAATGGCGATGCCTCCCCCGACtctcttgaaGTCGGGGG
AGGCATCGCCAGGG

Cells were transfected as above and selected with 2 μg/ml of puromycin.

In Vivo Ovarian Tumorigenicity Assays. Stable siRNA SKOV3 cells (1×10⁷) were injected into the left flank of female 6-8 week old BALB/c nu/nu mice. Tumor volume was assessed every week and determined by the formula (length×width×width×0.4). The mice were sacrificed 8 weeks after inoculation and tumors were excised for RNA, protein, and immunohistochemical analysis.

Prostate tumorigenicity Assay. Stable siRNA PC3M cells (1×10⁶) were injected into the left flank of female 6-8 week old BALB/c nu/nu mice. Tumor volume was assessed every week and determined by the formula (length×width×width×0.4). The mice were sacrificed 8 weeks after inoculation and tumors were excised for RNA, protein, and immunohistochemical analysis.

Direct Intratumoral Injection. PC3M or SKOV3 cells (1×10⁶) were injected into the left flank of female 6-8 week old BALB/c nu/nu mice. Three weeks after inoculation when the tumors had reached an average volume of 150-200 mm³, 50 μg of endotoxin-free plasmid DNA (Qiagen), pSUPER-luc or pSUPER-si-SV1 were injected directly into the tumor using a 28.5 gauge syringe (Becton Dickinson). Tumor volumes were measured twice weekly and calculated using the formula (length×width×width×0.4). Intratumoral tumor injections were repeated weekly for three weeks, at which time the mice were sacrificed and the tumors were excised for RNA analysis.

### Results and Discussion

RNA interference (RNAi) (pSUPER plasmid) was used to specifically decrease either wtKLF6 or splice variant expression. Each of these pSUPER-derived siRNAs specifically targets the respective KLF6 mRNA and protein, with no effect on the other isoforms. Three polyclonal cell line pools for each pSUPER-siRNA construct were generated and analyzed by qtRT-PCR and western blotting for KLF6 splicing., Stable si-wtKLF6 expression results in a 50% decrease in wild type KLF6 mRNA (** p<0.001), whereas stable si-SV1 and si-SV2 expression results in approximately a 60% decrease in KLF6 alternative splicing as measured by qtRT-PCR (** p<0.001).

The biological role of KLF6 and KLF6_{SV1} was also examined in ovarian cancer by altering their expression in the ovarian cancer cell line, SKOV3. Stable cell lines expressing siRNAs specific for luciferase (si-luc), wild type KLF6 (si-wtKLF6) and KLF6_{SV1} (si-sv1) were generated. After several polyclonal cell line pools from each siRNA infected construct were collected the level of decrease of both the mRNA and protein using qtRT-PCR and immunoblots was ascertained. Wild type KLF6 and KLF6_{SV1} message was reduced approximately 50%, while wtKLF6 and KLF6_{SV1} protein expression was decreased by 50% and 75% respectively.

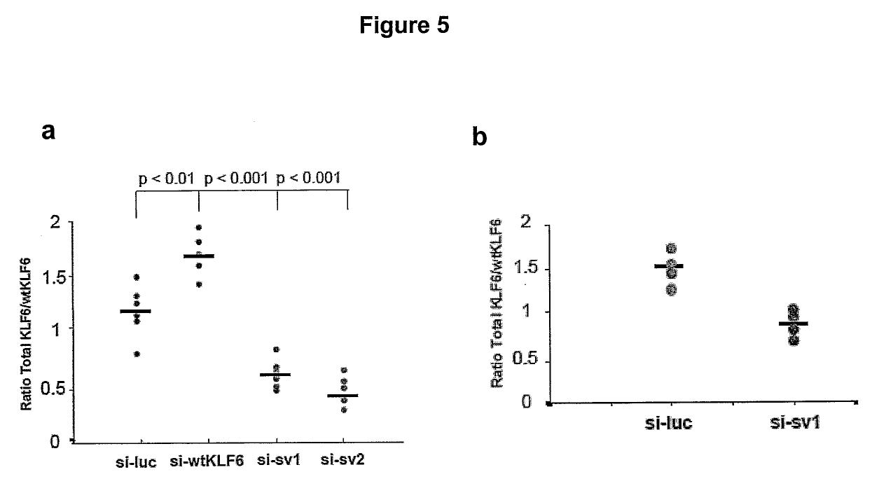

To determine if the siRNA-derived cell lines maintained their respective directed downregulation of KLF6 isoforms in vivo, the ratio of alternatively spliced KLF6 transcripts to wtKLF6 levels was quantified by qtRT-PCR from prostate tumor isolated RNA. The si-wtKLF6 derived tumors had 60% more alternative splicing relative to wild type KLF6 mRNA than did control luc tumors (p<0.01). The increase in the splice/wtKLF6 ratio seen in these tumors is similar to what is observed in metastatic prostate cancer samples. Additionally, tumors derived from the si-SV1 and si-SV2 cell lines had a 50% and 70% decrease in KLF6 alternative splicing respectively compared to the control tumors (p<0.001). (FIG. 5a)

PC3M and SKOV were also injected into nude mice and tumors allowed to form subcutaneously for three weeks prior to injecting pSUPER-si-SV1 plasmid DNA. Real time PCR confirmed that si-SV1-treated PC3M tumors had a significant reduction in KLF6 compared to luc controls injected tumors. (FIG. 5b)

### Example 6

### Preventing Metastasis Via Inhibition of KLF6 Splice Variants—Alteration of a Novel Binding Site

This example describes the identification and function of a novel binding site in the IVSΔA polymorphism.

### Materials and Methods

wtKLF6 and IVSΔA minigene constructs. See Examples 1 and 2 for relevant methodology.

SRp40 minigene constructs. The SRp40 and ASF constructs (generous gifts from Dr. A. Krainer) were previously described (Liu, et al., Genes Dev. 12:1998-2012 (1998)).

Cell culture. HeLa cells were obtained from the American Tissue Culture Collection (ATCC). Methods mirror those in Example 1.

RNA and qRT-PCR analysis. In addition to the methodology set forth in Example 2, primers for Example 6 include: SRp40 Forward: 5′-CCA AGG GAT GCA GAT GAT GCT G (SEQ ID NO: 28) and SRp40 Reverse 5′-GGA GCA TTT CGT CTA TCA TTT CGA-3′ (SEQ ID NO: 29).

Western Blot analysis. Methodology followed that as listed in Example 2. In addition, antibodies included SRp40 (kindly obtained as a gift from Adrian Krainer, Cold Spring Harbor Labs) and a T7 monoclonal (Novagen).

### Results and Discussion

One possible mechanism influencing alternative splicing, given the IVSΔA allele's proximity to the intron/exon boundary, was by generation or ablation of a splice site recognition sequence. Using the splicing enhancer motif prediction program ESEfinder (Cartegni, et al., Nucleic Acids Res. 2003; 31: 3568-71), three overlapping motifs for SR-protein splicing factor binding sites were identified. In the wtKLF6 sequence, high score motifs are present for SF2/ASF (2.61) and SRp55 (2.93). The IVSΔA allele abolishes these binding sites and generates a novel high-scoring SRp40 (4.30) motif (data not shown) with the sequence TCACATG.

The functional role of this predicted novel SRp40 site was tested by assessing the effect of incremental expression of SRp40 on alternative splicing in HeLa cells transfected with either the IVSΔA variant or wtKLF6 minigene constructs. SRp40 was detected by qtRT-PCR and Western blotting using a T7 monoclonal antibody. While co-expression did not result in a statistically significant increase in SRp40 mRNA or protein, SRp40 co-expression with IVSΔA did result in a dose-dependent increase in KLF6 splicing (increased total KLF6/wtKLF6 ratio) as compared to co-expression with the wtKLF6 minigene (** p<0.001, *** p<0.0001) (data not shown). Furthermore, coexpression of the SR protein ASF with the IVSΔA minigene construct did not increase KLF6 alternative splicing (data not shown).

### Example 7

### Preventing Metastasis Via Inhibition of KLF6 Splice Variants—Reduction in Splice Variant/KLF6 Binding

This example describes the interaction between wtKLF6 and the KLF6 splice variants. Specifically, data shows that the splice variants bind wtKLF6 and thus prevent wtKLF6 from functioning as a tumor suppressor gene. Therefore, any method to reduce the binding of these two proteins should result in a decrease in tumor formation and/or size.

### Materials and Methods

Cell culture and transient transfection. 293T cells were cultured as per Example 1.

KLF6 constructs. See Experiment 4 for methodology for KLF6 constructs. Additionally, wtKLF constructs were created with an additional HA tag.

Western Blot analysis. Protocols followed those set forth in Example 1. Western blotting was performed using monoclonal antibodies to HA (5B1D10, Zymed Labs).

Immunoprecipitation. All protein extracts were precleared by nutation in the presence of protein G-agarose beads and a corresponding control immunoglobulin for 30 min at 4° C. Supernatant was then collected, and 20-40 μL of either protein G-, HA-, or M2-FLAG-agarose beads were added for immunoprecipitation with nutation overnight at 4° C. of the desired protein. The next day, beads were washed four times with lysis buffer, and proteins were subsequently boiled off the beads in 1×SDS protein loading buffer and separated on SDS-PAGE gels for subsequent Western analysis. A monoclonal antibody to FLAG (F3165, Sigma) was used for immunoprecipitation.

### Results and Discussion

293T cells were transfected with the appropriate FLAG or HA tagged KLF6 or splice variant expression constructs. 24 hrs after transfection, protein extracts were harvested and immunoprecipitated as described above. Following cotransfection of HA-wtKLF6 and FLAG-KLF6SV1, a 38 kDa protein is evident. The same experiment was repeated with splice variant 2 and similar results were obtained. This data suggests that the KLF6_{SV1} and KLF6_{SV2} proteins interact at the protein level with full length wtKLF6.

### Example 8

### A Method for Diagnosis: The Extracellular Detection of Wild-Type KLF6 and KLF6 Splice Variants

This example describes the detection of secreted wtKLF6 and KLF6 splice variants and, thus, demonstrates a diagnostic method of this invention.

### Materials and Methods

Cell culture and transient transfection. See Example 1 for full methodology.

Western Blot analysis. See Example 2 for complete methodology.

wtKLF6 and SV FLAG constructs. See Example 4 for full methodology.

### Results and Discussion

KLF6 is markedly overexpressed in the cytoplasm of hepatocellular carcinoma in vivo, therefore the possibility that this accumulation is accompanied by extracellular release of either wtKLF6 or splice forms has been investigated. FLAG-tagged wtKLF6, KLF6_{SV1} or KLF6_{SV2} was transfected into PC3M cells. Cells were extensively washed, then medium was collected after 24 hours for analysis of KLF6 by Western blot following centrifugation to remove any cellular debris, and size exclusion chromatography to remove proteins <10 kDa. Western blot was performed on 30 ug of total protein using a monoclonal antibody to FLAG. There was significant detection of either wtKLF6 or splice forms in the medium but no expression in the vector transfected sample.

### Example 9

### Control of Tumor Growth Via Manipulation of Splice Variants

This example describes the relationship between KLF6 splice variants and tumor growth. Inhibition of the splice variants results in decrease in tumor formation, and causes tumor regression.

### Materials and Methods

pSUPER plasmid construction and transfection. See Example 5 for complete methodology.

Ovarian Tumorigenicity Assay. See Example 5 for complete methodology.

Prostate umorigenicity Assay. See Example 5 for complete methodology.

Direct Intratumoral Injection. See Example 5 for complete methodology.

RNA and qRT-PCR analysis. See Example 2 for relevant methodology.

### Results and Discussion

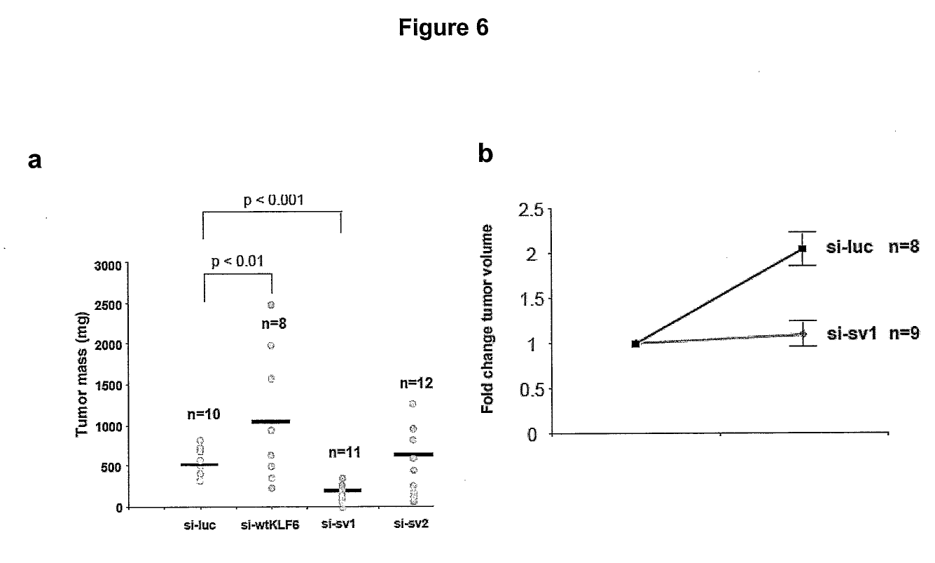

Decreased In Vivo Tumorigenicity of Stable Cell Lines Expressing KLF6_{SV1}-siRNA. Based on the marked functional differences between the various KLF6 siRNA cell lines, the affects of wtKLF6 or the KLF6_{SV1} and KLF6_{SV2} splice variants on tumorigenicity in vivo were examined. PC3M stable cell lines expressing specific siRNAs to either luciferase, wtKLF6, KLF6_{SV1} or KLF6_{SV2} were injected subcutaneously into nude mice and eight weeks after injection, the mice were sacrificed and tumor mass determined. Consistent with its role as a tumor suppressor gene in a number of human cancers, reduction of wtKLF6 mRNA resulted in over a 90% increase in tumorigenicity in vivo (p<0.01) (FIGS. 6a,b). In marked contrast, silencing of the KLF6_{SV1} transcript resulted in a 50% reduction in tumorigenicity in vivo (p<0.001). Targeted reduction of the KLF6_{SV2} transcript had no effect on in vivo tumorigenicity.

Intratumoral Administration of KLF6 si-SV1 Reduces Tumor Growth In Vivo. Intratumoral injection of naked plasmid DNA has been used successfully to direct expression of therapeutic proteins in a number of subcutaneous tumor xenograft models (Layergne E, et al. Cancer Res. 2003; 63: 7468-74; Igawa T, et al. Prostate. 2003; 55: 247-58). Based on the anti-tumorigenic effect of SV1-siRNA when stably expressed in PC3M cells prior to implantation, the effect of a plasmid expressing this si-SV1 on an established prostate tumor was examined. PC3M cells were injected into nude mice and tumors allowed to form subcutaneously prior to injecting pSUPER-si-SV1 plasmid DNA. pSUPER-si-SV1 treated tumors were completely growth inhibited, while pSUPER-si-luc injected tumors more than doubled in size (data not shown) (p<0.001).

Increased In Vivo Ovarian Tumor Growth Rate in Stable Cells Expressing si-wtKLF6. The role wtKLF6 or KLF6_{SV1} on ovarian tumor growth and metastasis in vivo was also examined. The rate of growth among the three groups of SKOV3 and PC3M transfected cells (control; n=9, wild type KLF6 n=15, or KLF6_{SV1} n=5) was altered considerably. In accordance with the role of a tumor suppressor gene, the reduction of wtKLF6 doubled the rate of subcutaneous tumor growth compared to si-luc tumors and on average developed 2-fold larger tumors (p<0.002; (data not shown). All mice were euthanized at 6 weeks when si-wtKLF6 mice developed severe abdominal swelling. Examination revealed that there was ascites accumulation and intraperitoneal carcinomatosis ( 9/9). Tumors in the si-wtKLF6 group were present on the peritoneal, intestinal and diaphragmatic surfaces ( 9/9). Average ascites volume was 600 uL, and expressed an average VEGF concentration of 870 pg/mL. Additionally, cells expressing si-SV1 were not tumorigenic in mice, tumors developed, but regressed after three weeks. No experiments could be performed on si-SV1 expression ovarian tumors due to their lack of growth.

### Example 10

### Control of Tumor Proliferation Via siRNA

This example describes a method to inhibit tumor proliferation via the use of siRNA. siRNA can be utilized to inhibit splice variant function, therefore reducing the proliferation induced by KLF6 splice variants. This method can lead to stabilization or even regression of tumors.

### Materials and Methods

KLF6 FLAG constructs. See Example 4 for a complete methodology of construct creation.

Western Blot analysis. See Example 2 for complete methodology. In addition, antibodies to c-myc, (SC-764, Santa Cruz Biotechnology), PCNA (Clone PC10, Dako) and rabbit polyclonal antibodies to p21 (H-164, Santa Cruz Biotechnology) were utilized.

pSUPER plasmid construction and transfection. See Example 5 for complete methodology.

Cell culture and transient transfection. SKOV3 and PC3M cells were cultured as stated previously in Examples 1, 2 and 5. Polyclonal pools of stable cell lines were generated by cotransfection of the pSUPER-si-luc, si-wtKLF6, si-SV1, or si-SV2 with a purpomycin expressing plasmid (See Example 5). Transfected cells were selected with 2 μg/ml of puromycin. For each construct, 3 independent polyclonal pools of stable cell line were generated and analyzed.

Tumorigenicity Assay. See Example 5 for complete methodology.

Direct Intratumoral Injection. See Example 5 for complete methodology.

Ovarian Tumorigenicity Assay. See Example 5 for complete methodology.

RNA and qRT-PCR analysis. See Example 2 for relevant methodology. Additional primers utilized include: ki-67 Forward: 5′-GAA GAG TTG TAA ATT TGC TTC T-3′ (SEQ ID NO: 30), ki-67 Reverse: 5′-ATG TTG TTT TGA CAC AAC AGG A-3′ (SEQ ID NO: 31), c-myc Forward: 5′-CAG CTG CTT AGA CGC TGG ATT T-3′ (SEQ ID NO: 32) and c-myc Reverse: 5′-ACC GAG TCG TAG TCG AGG TCA T-3′ (SEQ ID NO: 33).

Immunohistochemistry. Immunohistochemical staining for the expression of Factor VIII-related antigen and PCNA were carried out as previously described (Weidner, N. et al. Am J. Pathol. 1993; 143: 401; O'Reilly, M S, et al. Cell. 1997; 88: 277) using a factor VIII-related antigen (DAKO) antibody for the detection of tumor microvessel density and a PCNA antibody (Santa Cruz Biotechnology) for the detection of tumor cell proliferation. Briefly, microvessels that stained positive for factor VIII-related antigen were counted on 4 representative high power fields (400×) for each tumor. Data was expressed as the average number of microvessels per 400× field for each experimental tumor group. PCNA staining was determined by counting the number of positive cells per 400× field and dividing that number by the total number of cells in that particular field. For each tumor 4 high powered fields were counted and the average for each experimental tumor group was determined.

Analysis of proliferation. Proliferation was determined by estimating ³H-thymidine incorporation. For evaluation of the effects in prostate and ovarian tumor cells, PC3M and SKOV3 stable cell lines expressing either si-luc, si-wtKLF6, si-SV1, or si-SV2 were plated at a density of 50,000 cells/well in 12-well dishes. Forty eight hours after plating, 1 μCi/ml ³H-thymidine (Amersham) was added. After 2 hours, cells were washed 4 times with ice cold PBS and fixed in methanol for 30 min at 4° C. After methanol removal and cell drying, cells were solubilized in 0.25% sodium hydroxide/0.25% sodium dodecyl sulfate. After neutralization with hydrochloric acid (1N), disintegrations per minute were estimated by liquid scintillation counting.

### Results and Discussion

KLF6_{SV1} Inhibits p21 Activation. KLF6_{SV1} contains a novel 21 amino acid domain resulting from out-of-frame splicing of exon 3. To determine if this alternatively spliced form results in loss of function, an expression vector encoding KLF6_{SV1} was generated and analyzed for its ability to transactivate p21 in PC3M cells. In contrast to cells expressing the wtKLF6 minigene, the IVSΔA construct was unable to increase p21 RNA and protein levels to the same degree; resulting in 40% less p21 mRNA and 60% less p21 protein than cells expressing the wtKLF6 minigene. (** p<0.001, ANOVA, Bonferroni correction applied, n=4) (data not shown) To test the ability of the KLF6 variants to directly upregulate p21, the wtKLF6, KLF6_{SV1}, or KLF6_{SV2} expression plasmids were co-transfected with a p21 promoter reporter construct lacking functional p53 binding sites into 293T cells. Unlike wtKLF6, which transactivated the p21 promoter construct 6-fold (*** p<0.0001, ANOVA, Bonferroni correction applied, n=6), neither the KLF6_{SV1} nor KLF6_{SV2} proteins resulted in significant transactivation (*** p<0.0001, ANOVA, Bonferroni correction applied, n=9) (data not shown). In addition, whereas expression of wtKLF6 in 293T cells upregulated endogenous p21 three-fold, both variant proteins failed to upregulate p21 (data not shown).

To explore potential mechanisms underlying the splice variant-mediated reduction in tumorigenicity, the expression patterns of p21 were examined. Consistent with the previous findings, p21 mRNA and protein levels were increased over two-fold were increased in si-SV1 derived tumors (data not shown) (p<0.01).

KLF6_{SV1} Increases Tumor Cell Proliferation. Cells expressing the IVSΔA allele proliferate 30% more than the wtKLF6 expressing cells (** p<0.001, ANOVA, Bonferroni correction applied, n=9). Transfection with KLF6_{SV1} or KLF6_{SV2} expression vectors in the prostate cancer cell line PC3M resulted in no suppression of cell proliferation as measured by ³H-thymidine incorporation when compared to PC3M cells transfected with wtKLF6 (** p<0.001, ANOVA, Bonferroni correction applied, n=9).

Likewise, cell proliferation was increased by almost 90% in the si-wtKLF6 (*** p<0.001) and reduced by almost 60% cells in the si-SV1 cell lines (*** p<0.001) compared to the control cell line. No changes were noted in cell proliferation in the pSUPER-si-SV2 stable cell lines (data not shown).

Given the aforementioned results, and having identified correlations between KLF6 family members and clinical-pathological parameters in patient samples the proliferative status was examined in ovarian cancer cell line SKOV3 using siRNA to specifically downregulate splice variant expression.

In the si-wtKLF6 expressing SKOV3 cells, proliferation was increased approximately 2-fold (p<0.001) compared to the si-luc controls. Conversely, the cells with a reduction in KLF6_{SV1} exhibited a reduction of proliferation by approximately 40%.

Unexpectedly, given the previous findings in si-wtKLF6 and si-SV1 prostate cancer cell lines, but similar to results in non-small cell lung cancer, decrease in the KLF6 family members had no effect on p21 RNA and protein levels in the SKOV3 cells; suggesting a tissue/cancer specific effect (data not shown).

To determine whether the changes in proliferation observed in vitro were maintained in vivo, known proliferation markers were analyzed. Both the expression of c-myc and PCNA were consistently upregulated in ovarian tumors derived from the stable cell lines expressing si-wtKLF6 (data not shown).

In prostate tumors derived from Si-SV1 expressing cells, significantly less PCNA staining was exhibited (p<0.001) and a ˜30% reduction in ki-67 mRNA levels (p<0.0001), compared to control si-luc expressing tumors. (data not shown) In contrast, si-wtKLF6 tumors displayed higher PCNA staining in vivo (p<0.01), as well as a 50% increase in ki-67 mRNA level (data not shown).

### Example 11

### Control of Tumor Angiogenesis Via KLF6 Splice Variant siRNA

This example describes a method to inhibit tumor growth and metastasis via utilization of siRNA. siRNA can be utilized to inhibit splice variants, thereby resulting in a decrease in proteins which upregulate angiogenesis. Without proper vascular support, tumors cannot grow or metastasize. Thereby this method is useful as a method of treatment.

### Materials and Methods

Cell culture and transient transfection. SKOV3 (ovarian tumor) and PC3M cells were transfected with p-SUPER plasmids and cultured as stated previously in Example 10.

Ovarian Tumorigenicity Assay. See Example 5 for complete methodology.

Direct Intratumoral Injection. See Example 5 for complete methodology.

RNA and qRT-PCR analysis. See Examples 2 and 10 for relevant methodology. Primers include:

(SEQ ID NO: 34)

CDC2 Forward:
5′- AAC TAA GAA ACC ACT TTT CCA T -3′,

(SEQ ID NO: 35)

CDC2 Reverse:
5′- CAT TTT CAT CCA AGT TTT TGA C -3′,

(SEQ ID NO: 36)

Flt-1-F:
5′- GAG GAG GAT GAG GGT GTC TAT AGG T-3′,

(SEQ ID NO: 37)

Flt-1-R:
5′- GTG ATC AGC TCC AGG TTT GAC TT-3′,

(SEQ ID NO: 38)

VE-cadherin-F:
5′- TCC TCT GCA TCC TCA CTA TCA CA-3′,

(SEQ ID NO: 39)

VE-cadherin-R:
5′- GTA AGT GAC CAA CTG CTC GTG AAT-3′,

(SEQ ID NO: 40)

Ang2-F:
5′- TTA GCA CAA AGG ATT CGG ACA AT-3′,

(SEQ ID NO: 41)

Ang2-R:
5′- TTT TGT GGG TAG TAC TGT CCA TTC A-3′,

(SEQ ID NO: 42)

Tie-1-F:
5′- CAA GGT CAC ACA CAC GGT GAA-3′,

(SEQ ID NO: 43)

Tie-1-R:
5′- GCC AGT CTA GGG TAT TGA AGT AGG A-3′,
and

(SEQ ID NO: 44)

PECAM-F:
5′- GAG CCC AAT CAC GTT TCA GTT T-3′,

(SEQ ID NO: 45)

PECAM-R:
5′- TCC TTC CTG CTT CTT GCT AGC T-3′,

(SEQ ID NO: 46)

VEGF-Forward:
5′-CCG CAG ACG TGT AAA TGT TCC T-3′,
and

(SEQ ID NO: 47)

VEGF-Reverse:
5′- CGG CTT GTC ACA TCT GCA AGT A -3′.

Western Blot analysis. See Example 2 for complete methodology. In addition, antibodies to VEGF (SC-507, Santa Cruz Biotechnology), were utilized.

Immunohistochemistry. Tumor tissue was fixed for 4 hr in formalin and then embedded in paraffin according to standard histological procedures. Sections were deparaffinized with xylene followed by ethanol and then pretreated with 10 mM citrate buffer (pH 6.0) and incubated with rabbit antiserum against mouse CD31/PECAM for 2 hr.

### Results and Discussion

Targeted reduction of wtKLF6 and KLF6_{SV1} Alters the Expression and Secretion of VEGF

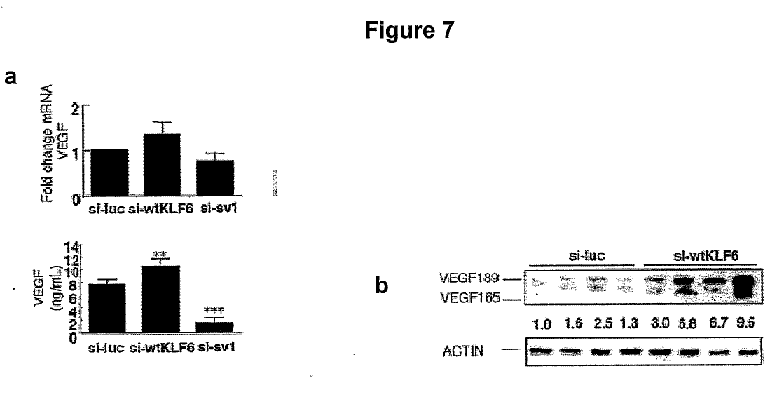

Given that angiogenesis is a key component of solid tumor growth and metastasis, the effects wtKLF6 reduction on angiogenesis and on one of the key angiogenic factors, VEGF was examined in ovarian tumor cell lines (SKOV3). Without angiogenesis tumor expansion cannot proceed beyond 1-2 mm because tumor proliferation is severely limited by nutrient supply to the tumor (Bamberger et al., Mol. Pathol. 2002; 55(6):348-59). Upon analysis of the expression and concentration of VEGF released into the conditioned media of the stable cell lines it was determined that reduction of wtKLF6 resulted in an increase in VEGF compared to control. (FIG. 7a) In marked contrast, the reduction of KLF6_{SV1} resulted in a dramatic decrease in the protein expression and concentration of VEGF secreted into the media, possibly explaining why these cells may not be tumorigenic in nude mice. (FIG. 7b)

Concordant with the in vitro studies, in vivo experiments demonstrated a statistically significant increase of VEGF both on the mRNA and protein levels in tumors with decreased expression of wtKLF6. On average the ovarian tumors with a reduction of wtKLF6 (n=11) had close to 8-fold higher concentrations of VEGF compared to si-luc control (data not shown).

To ascertain the effects of wtKLF6 on angiogenesis overall the expression of several angiogenic genes were examined, including Flt-1, KDR, PECAM-1, Tie1, VE-cadherin and Ang2. This profile has been shown to reflect angiogenesis better then any one marker alone. (Shih et al., Prostate Cancer Prostatic Dis. 2003; 6(2):131-7). Similar to the upregulation seen in VEGF, all six angiogenesis markers showed a significant increase in the tumors with decreased expression of wtKLF6 (data not shown)

PECAM-1/CD31 is highly expressed by endothelial cells, and is a reproducible marker of angiogenesis in transplanted prostate tumor models. Targeted reduction of the KLF6_{SV1} protein results in a 60% decrease in microvessel density as measured by the number of CD31/PECAM positive endothelial cells per 400× field, (p<0.0001) suggesting inhibition of angiogenesis in vivo. In addition, a panel of genes including RNAs for VEGF, Ang1, Ang2, Flt-1, KDR, Tie-1, VE-cadherin, and PECAM-1 was also examined. Consistent with the CD31 immunohistochemical data, expression of five angiogenic genes, Ang2, Flt-1, Tie-1, VE-cadherin, and PECAM/CD31 was significantly reduced in si-SV1 tumors (p<0.0001). Interestingly, VEGF, Ang1, and KDR were not significantly changed (not show) suggesting that the regulation of angiogenesis differs between different tumor types and tissues. Additionally, direct intratumoral injection of the pSUPER-si-SV1 plasmid resulted in a significant decrease in ki-67, PECAM, CDC2, Flt-1, and VE-cadherin mRNA levels compared to control luc treated tumors (*** p<0.0001).

### Example 12

### Control of Tumor Formation, Migration & Invasion Via siRNA

This example describes the method of tumor inhibition via utilization of siRNA to block tumor formation, migration and invasion. Presence of KLF6 splice variants results in upregulation of factors involved in tumor metastasis, migration and invasion. Therefore, utilization of siRNAs to block KLF6 splice variant function is a useful method to treat tumors.

### Materials and Methods

Cell culture and transient transfection. SKOV3 and PC3M cells were transfected with p-SUPER plasmids and cultured as stated previously in Example 10.

Colony Formation Assays. The ability of different transfectants to proliferate in an anchorage independent manner was quantified by the standard soft-agar assay. Approximately 10⁵ cells were resuspended in 2 ml of 0.4% (w/v) Noble agar (Difco) and overlaid on top of 1% (w/v) agar as described previously (Chan, A M. et al. Proc. Natl. Acad. Sci. U.S.A. 1994; 91: 7558-7562). After 3 weeks of incubation at 37° C., continuously growing colonies were visualized by staining with 1 mg/ml of p-iodonitotetrazonium violet. Colonies >2 mm in diameter were counted.

Migration and invasion assays. Standard invasion assays were performed in Boyden Chambers by using a reconstituted basement membrane (Matrigel™, 0.5 mg/ml) (Albini A, et al., Cancer Res: 1987; 47: 3239-45). Coated membranes were first blocked with 0.5% BSA in DMEM and equilibrated in 0.1% BSA/DMEM. Approximately 10⁵ cells in serum free DMEM were added to the upper chamber and conditioned medium derived from NIH 3T3 fibroblasts was used in the lower chamber as a chemoattractant. Following incubation for 19 hours at 37° C., cells in the upper chamber were thoroughly removed. Cells invaded through the barrier were fixed in 10% formalin and stained with DAPI in PBS. Nuclei were visualized under a fluorescence microscope and images of 5 randomly selected non-overlapping fields were counted.

### Results and Discussion

The effect of targeted reduction of KLF6 and its splice variants on a number of cancer phenotypes was examined. The ability of different KLF6 stable cell lines expressing siRNA to either luciferease (luc) wild type KLF6 (si-wt) or KLF6_{SV1} (si-SV1) to proliferate in an anchorage independent manner was quantified by soft-agar assay. Consistent with its function as a tumor suppressor gene, targeted reduction of wtKLF6 results in a 30% increase in the number of all colonies formed (* p<0.01) and a 90% in large colony formation compared to the si-luc control cell line (*** p<0.0001). Furthermore, targeted of KLF6_{SV1} leads to a 50% reduction in both the number of all colonies and large colonies formed compared to controls (** p<0.001).

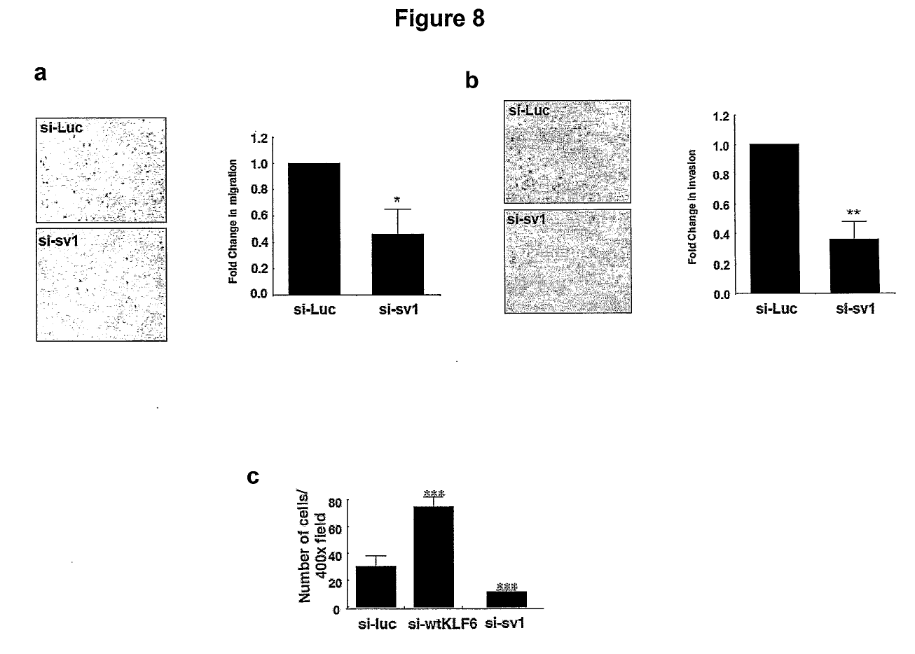

Given the role of cell migration and invasion in the progression of localized cancer to disseminated disease, these traits were analyzed in PC3M siRNA cell lines. Targeted reduction of the KLF6_{SV1} protein resulted in a 60% decrease in both cell migration (p<0.01) and invasion (p<0.001) of this highly metastatic cell line (FIGS. 8a,b). Reduction of the wtKLF6 or KLF_{SV2} proteins had no effect on either cell migration or invasion, (data not shown).

The invasive capacity of SKOV3 cells depleted of either wtKLF6 or KLF6_{SV1} was also explored. Strikingly, targeted reduction of wtKLF6 resulted in a 70% (2.5 fold, p<0.001) increase in cellular invasion, whereas, down regulation of KLF6_{SV1} revealed a 60% reduction in invasion. (FIG. 8c)

Taken together, these findings suggest that the down-regulation of wtKLF6 combined with increased expression of the KLF6_{SV1} protein seen in metastatic patient samples and in the PC3M and SKOV3 cell lines may play a role in the invasive capacity of a tumor and its ability to metastasize.

### Example 13

### Regulation of E Cadherin and Prevention of Tumor Metastasis

This example describes a method for inhibition of tumor metastasis via the utilization of siRNA. KLF6 splice variants are able to alter the levels of E-cadherin, which allows them to mobilize and metastasize. Inhibition of KLF6 splice variants therefore, can be useful in prevention of tumor metastasis as tumors that have elevated levels of E-cadherin are not metastatic.

### Materials and Methods

KLF6 and E-cadherin constructs. KLF6 constructs were created as described in Example 4. Additionally, E-Cadherin constructs were created as follows. Genomic DNA extracts from the 293T cell line were PCR amplified using E-cadherin promoter primer pairs. PCR products were purified (Qiagen) and the promoter fragments were cloned into the luciferase reporter pGL3 basic (Promega). All promoter reporter constructs were sequenced prior to transfection.

Western Blot analysis. See Examples 2 and 10 for complete methodology. In addition, antibodies to E-cadherin (SC-7870) and Beta-catenin (SC-7963) were obtained from Santa Cruz Biotechnology. c-myc antibody was purchased from Oncogene (OP10) and PCNA antibody was obtained from DAKO (M-0897). Enhanced chemiluminescent immunoblot images were analyzed by scanning densitometry as described above.

RNA and qRT-PCR analysis. See Examples 2 for relevant methodology. Primers include—E-cadherin-Forward: 5′-CAA AGT GGG CAC AGA TGG TGT G-3′ (SEQ ID NO: 48) and E-cadherin-Reverse: 5′-CTG CTT GGA TTC CAG AAA CGG-3′ (SEQ ID NO: 49).

Cell culture and transient transfection. SKOV3 cells were cultured and transfected as stated previously in Examples 1 and 5.

Immunohistochemistry. Immunohistochemical staining for the expression of B-catenin was carried out as described in Example 10.

Luciferase transactivation. Luciferase transactivation assays were performed 24 hours after transfection into 293T cells plated at 100,000 cells/well in 12-well dishes with DNA containing 1.5 μg E-cadherin promoter constructs and either 1.5 μg pCI-neo-KLF6, pCI-neo-KLF6SV1, or pCIneo-KLF6_{SV2}. The TK promoter-Renilla Luciferase construct (Promega), 2 ng, was used to normalize each transfection experiment.

Sequence and Genotype Analysis. See Examples 1 and 2 for relevant methodology.

HeLA cell transfection. HeLa cells were purchased from the American Tissue Culture Collection (ATCC). Stable cell lines were generated by cotransfection of the pSUPER-si-Luc, pSUPER-si-wtKLF6, and pSUPER-si-SV1 with a puromycin dependent plasmid as described herein.

Chromatin immunoprecipitation (ChIP) analysis. Chromatin immunoprecipitation (ChIP) analysis was performed as previously described in detail⁴⁵. Antisera against histone H3 and anti-acetyl (K9) histone H3 were purchased (Upstate Biotech). Oligonucleotide primers used for the PCR are as follows. Site 1: forward 5′-GAC TACAGG CGC CCA CCA CCA-3′ (SEQ ID NO: 52), reverse 5′-TGT GGG ACT CCC ATA CAA TTA AAA-3′ (SEQ ID NO: 53); site 2: forward 5′-GCC CCG ACT TGT CTC TCT ACA A-3′ (SEQ ID NO: 54), reverse 5′-TGG AGA TGG GGT CTC ACT CTT TC-3′ (SEQ ID NO: 55); Site 3: forward 5′-GTC TTA GTG AGC CAC CGG CGG G-3′ (SEQ ID NO: 56), reverse 5′-GTT CAC CTG CCG GCC ACA GCC-3′ (SEQ ID NO: 57); Site 4: forward 5′-GCG GTA CGG GGG GCG GT-3′ (SEQ ID NO: 58), Reverse 5′-ACG CCG AGC GAG GGC AGG CG-3′ (SEQ ID NO: 59).

### Results and Discussion

### E-Cadherin is a Novel Target of KLF6

In order for a cell to achieve dissociation and lose its epithelial phenotype, cells must generate a disruption of the direct cadherin-cadherin interaction between the cells. One way to reduce the strength of the cell adherens junction is to decrease the amount of E-cadherin protein available in the cell, which occurs in many cancers. Given the importance of E-cadherin in maintaining cell-cell adhesion whether the alteration of E-cadherin contributed to the morphological changes observed in stable cell lines was examined. qtRT-PCR and western blots were performed to assess any differences in E-cadherin expression. Strikingly, a 50% reduction in E-cadherin mRNA expression in the si-wtKLF6 cells (2.5 fold, p<0.01) and an approximate 5-fold upregulation in the si-sv1 cells (p<0.0001) compared to si-luc controls was observed. The protein levels also displayed similar alterations (data not shown).

The reduction of E-cadherin also causes an imbalance in the amount of β catenin in the cytoplasm since the interaction between E-cadherin and β catenin is necessary for cell-cell adhesion and when β catenin is not bound to E-cadherin it is phosphorylated and targeted for degradation. An accumulation of β catenin may interfere with its phosphorylation and an excess of unphosphorylated β catenin can translocate to the nucleus were it interacts with the LEF transcription factor family, leading to the activation of several genes, such as cyclin D1, c-myc and matrilysin. (Polakis, Genes Dev. 2000; 14(15):1837-51).

Given the dramatic changes in the expression of E-cadherin, the subcellular localization of β catenin and the expression of its downstream target c-myc were examined. After step-wise separation of cytoplasmic and nuclear extracts from each of the stable cell lines the protein levels of β catenin in the cytoplasmic extracts were measured. The reduction of wtKLF6 resulted in an 80% decrease of cytoplasmic β catenin, conversely the reduction of KLF6_{SV1} displayed a 2.5 fold upregulation of β catenin in the cytoplasm. PCNA, which resides solely in the nucleus, was used as a marker to determine the compartmentalization of the nuclear and cytoplasmic extracts. As a result of the accumulation of β catenin in the cytoplasm, a 70% decrease in c-myc protein expression in si-sv1 compared to si-luc control was observed (data not shown).

Differences in the translocation of β catenin from the cytoplasm to the nucleus were also measured by immunocytochemistry in the stable cell lines. Each cell line was incubated with both a monoclonal β catenin antibody and DAPI, as a nuclear stain, to ascertain the number of cells with either nuclear or cytoplasmic β catenin. The findings supported what was observed in the nuclear/cytoplasmic fractionation studies. A statistically significant increase in the percentage of cells with positive nuclear β catenin in the si-wtKLF6 stable cell lines compared to si-luc was observed (43 from 28). Inversely, in the si-sv1 stable cells there was a statistically significant decrease in the number of cells with nuclear β catenin. (14 from 28) These findings may contribute to both the decrease in proliferation and the epithelial-to-mesenchymal transition observed when wtKLF6 is decreased.

Sp1-like proteins and Kruppel-like factors (KLFs) are highly related zinc-finger proteins that regulate a large number of the genes that have GC-rich promoters. (Kaczynski et al., Genome Biol. 2003; 4(2):206). Four deletion constructs of the E-cadherin promoter were utilized to test the ability of KLF6 and KLF6_{SV1} to bind to the four putative Sp1 sites and GC-rich regions approximately 500 bp upstream from the initiation start site of the E-cadherin gene. The full-length construct which retained all four GC-rich regions was activated 2-fold by wtKLF6 and remains unchanged in the presence of KLF6_{SV1} in 293T cells relative to the insertless pCI-Neo expression vector. Titrating the amount of KLF6 transfected into 293T cells revealed a dose-dependent trans-activation of the full-length E-cadherin promoter, ranging from 1.5-fold to 3-fold as DNA increased from 1 μg to 2 μg (data not shown).

The −435 and −230 construct had the strongest response to wtKLF6 which suggested that the three Sp1 sites approximately 500 base pairs upstream from the initiation site were essential for full activation. Unlike what was seen for the Wt1 activation of the E-cadherin promoter, where the GC-rich region—67 base pairs from the start site were essential for binding, that site alone did not reveal any activation in the presence of wtKLF6. (Hosono et al., J Biol. Chem. 2000; 275(15):10943-53). Additionally, the KLF6_{SV1} cDNA did not show trans-activation in the presence of all constructs which is reasonable for the lack of the DNA-binding domain in this isoform.

Given the direct trans-activation of the E-cadherin promoter by wtKLF6 and not the KLF6_{SV1} variant, whether decreased expression of wtKLF6 and upregulation of the splice variants would correlate with a decrease in E-cadherin expression in patient EOC tumors was examined. Notably, 16 EOC tumors with high KLF6 splicing index displayed an approximate 80% reduction in E-cadherin expression compared to 17 tumors with low KLF6 splicing. Since the reduction of E-cadherin is hypothesized to contribute to the spread and progression of EOC tumors, whether stage 1V tumors with a high splicing index showed a significantly greater reduction in the mRNA expression of E-cadherin compared to stage III tumors was also examined. (Freedman et al., J Transl Med. 2004; 2(1):23). Strikingly, stage 1V tumors with high splicing had minimal E-cadherin expression (almost none) and the stage III tumors had a reduction of approximately 80% compared to tumors with low splicing, suggesting that less differentiated, higher grade tumors that had increased KLF6_{SV1} had a greater reduction in E-cadherin.

Additionally, to detect in vivo occupation of the E-cadherin promoter by KLF6, ChIP analysis was performed. HeLa cells were transfected with a mammalian expression vector for KLF6 and it was determined that KLF6 occupied two putative target sites, site 3, at −150 and −117 from the transcriptional start site (data not shown), A strong signal was also detected within site 4, which corresponds to the KLF6 target site in the (−103)-luciferase reporter construct. Despite the fact that sequences overlapping site 4 failed to potentiate the transactivation of luciferase activity by KLF6, ChIP analysis revealed that occupancy within the E cadherin locus by KLF6 at site 3 indicated the inability for the ChIP study to discriminate between the two closely linked sequences within 500 base pairs. However, the two upstream regions, containing putative KLF6 sites, failed to generate a detectable signal and point to KLF6 selectivity within the E-cadherin locus. Furthermore, ChIP analysis indicated the level of lysine acetylation of histone H3 at lysine 9 (K9), an epigenetic modification directly associated with gene activation, clearly overlaps with KLF6 occupation, and supports a model for transcriptional activation of E cadherin by KLF6 within this genomic region. These data support the luciferase data and further define E-cadherin as a novel target of KLF6.

### Example 14

### KLF6 Allelic Loss is a Frequent Event Defining Ovarian Cancer Progression and Metastatic Spread

### Methods

Tumor sample preparation and DNA isolation. EOC tumor specimens were collected and analyzed as described above in Example 2.

LOH and DNA mutation analysis. Fluorescent LOH analysis using genomic DNA from matched normal/tumor ovarian tissue and markers has been previously described (Narla et al., 2001, supra). The exponential range of the PCR was determined for each marker and sample, and was between 30-38 cycles. Data were analyzed using ABI Genescan and Genotyper software packages (Perkin Elmer) and allelic loss was scored by two independent observers. A relative allele ratio of less than 0.7, correlating with an allele loss of approximately 40%, was defined as LOH5. All sample marker combinations were analyzed at least twice. PCR products were directly sequenced as described in Example 1.

### Results

KLF6 loss of heterozygosity (LOH) and decreased expression in EOC. The clinical-pathologic profiles of these samples (Table 1) is representative of the varied, clinical spectrum of EOC wherein FIGO stage III, grade 3, serous ovarian carcinomas dominate as the most frequent presentation of advanced EOC. A total of 68 paired EOC and normal samples were assayed for LOH using six microsatellite markers: KLF6M1, KLF6M2, and KLF6M4 which tightly flank the KLF6 gene locus (Narla et al., 2001, supra) and D10S249, D10S594 and D10S591 which are more distal. This marker set rendered all tumor samples informative at the KLF6 locus.

TABLE 1
Epithelial:

| | |
|---|---|
| Serous carcinoma | 46 (68%) |
| Endometrioid | 8 (12%) |
| Mucinous carcinoma | 9 (13%) |
| Clear cell | 5 (7%) |

Histological grade

| | |
|---|---|
| 1 | 5 (7%) |
| 2 | 19 (28%) |
| 3 | 44 (65%) |

FIGO stage

| | |
|---|---|
| I | 7 (10%) |
| II | 4 (6%) |
| III | 39 (57%) |
| IV | 18 (26%) |

Primary residual tumor

| | |
|---|---|
| None | 15 (22%) |
| ? 2 cm | 20 (29%) |
| >2 cm | 19 (28%) |
| No data | 14 (21%) |

Overall, 54% ( 37/68) of the EOC samples demonstrated LOH of KLF6 locus. LOH was subtype specific, p<0.02, (Table 2): allelic loss was present in 70% of serous carcinomas (32 out of 46), 50% of the endometriod tumors (4 out of 8), and 11% of the mucinous EOCs (1 out of 9). None of the 5 clear cell carcinomas demonstrated LOH (Table 2). In addition, KLF6 LOH was significantly correlated with tumor FIGO stage (p<0.025) and grade (p<0.05).

TABLE 2
Summary of allelic loss of KLF6 in epithelial ovarian cancer (EOC)

| Histologic | | Allelic loss of 10p15 |
|---|---|---|
| Serous | 32/46 | (70%)* |
| Endometrioid | 4/8 | (50%) |
| Mucinous | 1/9 | (11%) |
| Clear cell | 0/5 | (0%) |
| Total | 37/68 | (54%) |

*Allelic loss of KLF6 was significantly more frequently found in serous carcinoma (p ≦ 0.02)

KLF6 mutation status was examined by direct sequencing exon 2 in all samples. This exon encodes three quarters of the wild-type protein and contains the majority of mutations previously identified in other human cancers. In clear contrast to the previous findings in prostate, colorectal, and hepatocellular carcinomas, no mutations were detected in any of the EOC samples analyzed.

It was then determined whether allelic loss was associated with decreased KLF6 mRNA expression. Eleven of the 26 tumor samples demonstrating LOH and with RNA available for analysis had on average an approximate 80% reduction in expression when compared to tumors without LOH (p<0.0007) or when compared to normal ovary tissue (p<0.003). KLF6 expression levels in tumors without LOH trended lower but were not statistically significantly different from normal whole ovary tissue. These findings are in accord with previous studies in non-small cell lung cancer where in 14/14 samples with LOH displayed a down-regulation in KLF6 mRNA expression (Ito et al., Cancer Res. 2004; 64, 3838-43).

These results demonstrate that LOH is another mechanism by which KLF6 can be dysregulated, independent of any SNPs or other inactivating mutations.

### Example 15

### KLF6 Gene Splicing is Disregulated in Ovarian Tumors

It was explored whether KLF6 alternative splice variants were produced in ovarian tissue and whether or not KLF6 alternative splicing was disregulated in EOC. RNA from 33 tumors and a panel of five normal ovarian tissue samples was analyzed by qRT-PCR. All but one of the 33 tumor samples analyzed, 32/33 (97%), revealed evidence of KLF6 alternative splicing.

It was next determined the levels of each KLF6 isoform using highly specific and sensitive sets of real time PCR primers. All tumors expressed detectable levels of KLF6-SV1 whereas 25/33 (75%) expressed KLF6-SV2. KLF6-SV3 was not detected. In our sample set of tumors, KLF6-SV1 expression was increased on average 4-fold, being greatest in tumors with LOH, while KLF6-SV2 expression was not significantly different. This KLF6-SV1 increase is particularly striking since wtKLF6 expression was decreased in tumors.

### Example 16

### KLF6 Splice Variant Overexpression Correlates with Advanced Tumor Grade

A possible correlation between increased KLF6 alternative splicing and EOC tumor grade was explored. First, to quantitatively assess the relative amount of splice variants in each tumor sample, the “KLF6 splicing index”: the ratio of all KLF6 transcripts compared to only wtKLF6 was determined. qRT-PCR primers specific to either conserved or unique regions within the KLF6 transcript were used such that only wtKLF6 or both wt and variant transcripts (total) were amplified (Narla et al, Cancer Res., 65:5761-8, 2005; Narla et al., Cancer Res., 65:1213-22, 2005). Second, because the splice variants are mislocalized to the cytoplasm (Narla et al, Cancer Res., 65:5761-8, 2005; Narla et al., Cancer Res., 65:1213-22, 2005), splicing indices with KLF6 staining patterns were compared. A KLF6 monoclonal antibody was used to detect both wt and splice KLF6 staining intensity and localization in 41 ovarian tumors. Interestingly, all positive staining was localized to the cytoplasm with no detectable nuclear staining. Moreover, splicing ratios and staining intensities were positively correlated (p≦0.001). In addition, to determine relative contribution of staining by each isoform, the levels of KLF6-SV1 and -SV2 were compared. There was an approximate 2-fold increase in KLF6-SV1 mRNA expression in tumors with high cytoplasmic staining (p≦0.004). Compared to SV1, KLF6-SV2 was expressed at low levels in 75% of samples and remained unchanged regardless of staining intensity.

Finally, a 2-fold increase in KLF6 splicing index in poorly differentiated grade III tumors compared to well to moderately differentiated grade I or II tumors (p≦0.035) was detected. Similarly, 18/19 tumors with high KLF6 staining patterns were grade III tumors. Low staining patterns were not as informative as only 4/12 tumors were stage II. Nonetheless, this staining/grade distribution pattern was statistically significant (p≦0.05). Taken together, these findings suggested that increased KLF6 splice form expression, primarily SV1, is associated with more aggressive and poorly differentiated tumors.

The present invention is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims.

It is further to be understood that all values are approximate, and are provided for description.

Patents, patent applications, procedures, and publications cited throughout this application are incorporated herein by reference in their entireties.

1-35. (canceled)

36. A method of determining cancer progression in a subject diagnosed with a tumor by determining the presence of a KLF6 splice variant in a tumor cell.

37. The method of claim 36, wherein the method comprises determining the presence of a KLF6 splice variant nucleic acid.

38. The method of claim 36, wherein the method comprises determining the presence of a KLF6 splice variant polypeptide.

39. The method of claim 37 wherein the KLF6 splice variant is KLF6 splice variant-1 (KLF6_{SV1}).

40. A method of determining tumor stage in a subject diagnosed with a tumor by determining the ratio between a wild-type KLF6 and a KLF6 splice variant in a tumor cell, wherein a low ratio of wild-type KLF6 to the KLF6 splice variant, is indicative of an advanced tumor stage.

41. The method of claim 40, wherein the KLF6 splice variant is KLF6 splice variant-1 (KLF6_{SV1}).

42. The method of claim 41, wherein the tumor is a prostate tumor.

43. The method of claim 41, wherein the tumor is an ovarian tumor.

44. The method of claim 41, wherein the tumor is a liver hepatocellular carcinoma tumor.

45. A method of determining cancer progression in a subject diagnosed with a tumor by determining the ratio between a wild-type KLF6 and a KLF6 splice variant in a tumor cell, wherein a decrease in the ratio of wild-type KLF6 to the KLF6 splice variant, relative to the ratio between wild-type KLF6 and a KLF6 splice variant measured on a previous occasion, is indicative of cancer progression.

46. The method of claim 45, wherein the KLF6 splice variant is KLF6 splice variant-1 (KLF6_{SV1}).

47. The method of claim 45, wherein the tumor cell is from a hormone-dependent tumor.

48. The method of claim 47, wherein the tumor is a prostate tumor.

49. The method of claim 47 wherein the tumor is an ovarian tumor.

50. The method of claim 44, wherein the tumor is a liver hepatocellular carcinoma tumor.

51-69. (canceled)

70. A method of determining cancer progression in a subject diagnosed with a tumor determining the presence of wild-type KLF6, or a KLF6 splice variant, from the extracellular medium surrounding the tumor.

71. The method of claim 70, wherein the cancer progression is metastasis.

72-79. (canceled)


SEQUENCE LISTING (60 sequences)

SEQ ID NO: 1 - DNA, 588 bp, Homo sapiens
atggacgtgctccccatgtgcagcatcttccaggagctccagatcgtgcacgagaccggctacttctcggcgctgccgtctctggaggagtactggcaacagacctgcctagagctggaacgttacctccagagcgagccctgctatgtttcagcctcagaaatcaaatttgacagccaggaagatctgtggaccaaaatcattctggctcgggagaaaaaggaggaatccgaactgaagatatcttccagtcctccagaggacactctcatcagcccgagcttttgttacaacttagagaccaacagcctgaactcagatgtcagcagcgaatcctctgacagctccgaggaactttctcccacggccaagtttacctccgaccccattggcgaagttttggtcagctcgggaaaattgagctcctctgtcacctccacgcctccatcttctccggaactgagcagggaaccttctcaactgtggggttgcgtgcccggggagctgccctcgccagggaaggagaaaagccttacagatgctcatgggaagggtgtgagtggcgttttgcaagaagtgatgagttaa

SEQ ID NO: 2 - protein, 195 aa, Homo sapiens
MDVLPMCSIFQELQIVHETGYFSALPSLEEYWQQTCLELERYLQSEPCYVSASEIKFDSQEDLWTKIILAREKKEESELKISSSPPEDTLISPSFCYNLETNSLNSDVSSESSDSSEELSPTAKFTSDPIGEVLVSSGKLSSSVTSTPPSSPELSREPSQLWGCVPGELPSPGKEKSLTDAHGKGVSGVLQEVMS

SEQ ID NO: 3 - DNA, 726 bp, Homo sapiens
atggacgtgctccccatgtgcagcatcttccaggagctccagatcgtgcacgagaccggctacttctcggcgctgccgtctctggaggagtactggcaacagacctgcctagagctggaacgttacctccagagcgagccctgctatgtttcagcctcagaaatcaaatttgacagccaggaagatctgtggaccaaaatcattctggctcgggagaaaaaggaggaatccgaactgaagatatcttccagtcctccagaggacactctcatcagcccgagcttttgttacaacttagagaccaacagcctgaactcagatgtcagcagcgaatcctctgacagctccgaggaactttctcccacggccaagtttacctccgaccccattggcgaagttttggtcagctcgggaaaattgagctcctctgtcacctccacgcctccatcttctccggaactgagcagggaaccttctcaactgtggggttgcgtgcccggggagctgccctcgccagggaaggtgcgcagcgggacttcggggaagccaggagaaaagccttacagatgctcatgggaagggtgtgagtggcgttttgcaagaagtgatgagttaaccaggcacttccgaaagcacaccggggccaagccttttaaatgctcccactgtgacaggtgtttttccaggtctgaccacctggccctgcacatgaagaggcacctctga

SEQ ID NO: 4 - protein, 241 aa, Homo sapiens
MDVLPMCSIFQELQIVHETGYFSALPSLEEYWQQTCLELERYLQSEPCYVSASEIKFDSQEDLWTKIILAREKKEESELKISSSPPEDTLISPSFCYNLETNSLNSDVSSESSDSSEELSPTAKFTSDPIGEVLVSSGKLSSSVTSTPPSSPELSREPSQLWGCVPGELPSPGKVRSGTSGKPGEKPYRCSWEGCEWRFARSDELTRHFRKHTGAKPFKCSHCDRCFSRSDHLALHMKRHL

SEQ ID NO: 5 - DNA, 728 bp, Homo sapiens
atggacgtgctccccatgtgcagcatcttccaggagctccagatcgtgcacgagaccggctacttctcggcgctgccgtctctggaggagtactggcaacagacctgcctagagctggaacgttacctccagagcgagccctgctatgtttcagcctcagaaatcaaatttgacagccaggaagatctgtggaccaaaatcattctggctcgggagaaaaaggaggaatccgaactgaagatatcttccagtcctccagaggacactctcatcagcccgagcttttgttacaacttagagaccaacagcctgaactcagatgtcagcagcgaatcctctgacagctccgaggaactttctcccacggccaagtttacctccgaccccattggcgaagttttggtcagctcgggaaaattgagctcctctgtcacctccacgcctccatcttctccggaactgagcagggaaccttctcaactgtggggttgcgtgcccggggagctgccctcgccagggaaggtgcgcagcgggacttcggggaagccaggtgacaagggaaatggcgatgcctcccccgacggcaggaggagggtgcaccggtgccactttaacggctgcaggaaagtttacaccaaaagctcccacttgaaagcacaccagcggacgcacacaggtgtttttccaggtctgaccacctggccctgcacatgaagaggcacctctga

SEQ ID NO: 6 - protein, 237 aa, Homo sapiens
MDVLPMCSIFQELQIVHETGYFSALPSLEEYWQQTCLELERYLQSEPCYVSASEIKFDSQEDLWTKIILAREKKEESELKISSSPPEDTLISPSFCYNLETNSLNSDVSSESSDSSEELSPTAKFTSDPIGEVLVSSGKLSSSVTSTPPSSPELSREPSQLWGCVPGELPSPGKVRSGTSGKPGDKGNGDASPDGRRRVHRCHFNGCRKVYTKSSHLKAHQRTHTGVFPGLTTWPCT

SEQ ID NO: 7 - DNA, 25 bp, Homo sapiens
cgggcagcaatgttatctgtccttc

SEQ ID NO: 8 - DNA, 24 bp, Homo sapiens
ttctgaggctgaaacatagcaggg

SEQ ID NO: 9 - DNA, 21 bp, Homo sapiens
ttgcagtcagtccggtgtttg

SEQ ID NO: 10 - DNA, 25 bp, Homo sapiens
ggtgctatgccgcttcttacaggac

SEQ ID NO: 11 - DNA, 29 bp, Homo sapiens
gtcatggcaatcacatgccttctctggtt

SEQ ID NO: 12 - DNA, 29 bp, Homo sapiens
aaccagagaaggcatgtgattgccatgac

SEQ ID NO: 13 - DNA, 20 bp, Homo sapiens
cggacgcacacaggagaaaa

SEQ ID NO: 14 - DNA, 19 bp, Homo sapiens
cggtgtgctttcggaagtg

SEQ ID NO: 15 - DNA, 19 bp, Homo sapiens
ctgccgtctctggaggagt

SEQ ID NO: 16 - DNA, 22 bp, Homo sapiens
tccacagatcttcctggctgtc

SEQ ID NO: 17 - DNA, 20 bp, Homo sapiens
caatgaccccttcattgacc

SEQ ID NO: 18 - DNA, 20 bp, Homo sapiens
gatctcgctcctggaagatg

SEQ ID NO: 19 - protein, 21 aa, Homo sapiens
EKSLTDAHGKGVSGVLQEVMS

SEQ ID NO: 20 - DNA, 66 bp, Homo sapiens
gatccccaggcttttctccttccctggcttcaagagagccagggaaggagaaaagccttttggaaa

SEQ ID NO: 21 - DNA, 67 bp, Homo sapiens
agcttttccaaaaaggcttttctccttccctggctctcttgaagccagggaaggagaaaagcctggg

SEQ ID NO: 22 - DNA, 64 bp, Homo sapiens
gatccccgccaggagaaaagccttacttcaagagagtaaggcttttctcctggctttttggaaa

SEQ ID NO: 23 - DNA, 64 bp, Homo sapiens
agcttttccaaaaagccaggagaaaagccttactctcttgaagtaaggcttttctcctggcggg

SEQ ID NO: 24 - DNA, 64 bp, Homo sapiens
gatcccctacttcgaaatgtccgttcttcaagagagaacggacatttcgaagtatttttggaaa

SEQ ID NO: 25 - DNA, 64 bp, Homo sapiens
agcttttccaaaaatacttcgaaatgtccgttctctcttgaagaacggacatttcgaagtaggg

SEQ ID NO: 26 - DNA, 64 bp, Homo sapiens
gatcccctggcgatgcctcccccgacttcaagagagtcgggggaggcatcgccatttttggaaa

SEQ ID NO: 27 - DNA, 64 bp, Homo sapiens
agcttttccaaaaatggcgatgcctcccccgactctcttgaagtcgggggaggcatcgccaggg

SEQ ID NO: 28 - DNA, 22 bp, Homo sapiens
ccaagggatgcagatgatgctg

SEQ ID NO: 29 - DNA, 24 bp, Homo sapiens
ggagcatttcgtctatcatttcga

SEQ ID NO: 30 - DNA, 22 bp, Homo sapiens
gaagagttgtaaatttgcttct

SEQ ID NO: 31 - DNA, 22 bp, Homo sapiens
atgttgttttgacacaacagga

SEQ ID NO: 32 - DNA, 22 bp, Homo sapiens
cagctgcttagacgctggattt

SEQ ID NO: 33 - DNA, 22 bp, Homo sapiens
accgagtcgtagtcgaggtcat

SEQ ID NO: 34 - DNA, 22 bp, Homo sapiens
aactaagaaaccacttttccat

SEQ ID NO: 35 - DNA, 22 bp, Homo sapiens
cattttcatccaagtttttgac

SEQ ID NO: 36 - DNA, 25 bp, Homo sapiens
gaggaggatgagggtgtctataggt

SEQ ID NO: 37 - DNA, 23 bp, Homo sapiens
gtgatcagctccaggtttgactt

SEQ ID NO: 38 - DNA, 23 bp, Homo sapiens
tcctctgcatcctcactatcaca

SEQ ID NO: 39 - DNA, 24 bp, Homo sapiens
gtaagtgaccaactgctcgtgaat

SEQ ID NO: 40 - DNA, 23 bp, Homo sapiens
ttagcacaaaggattcggacaat

SEQ ID NO: 41 - DNA, 25 bp, Homo sapiens
ttttgtgggtagtactgtccattca

SEQ ID NO: 42 - DNA, 21 bp, Homo sapiens
caaggtcacacacacggtgaa

SEQ ID NO: 43 - DNA, 25 bp, Homo sapiens
gccagtctagggtattgaagtagga

SEQ ID NO: 44 - DNA, 22 bp, Homo sapiens
gagcccaatcacgtttcagttt

SEQ ID NO: 45 - DNA, 22 bp, Homo sapiens
tccttcctgcttcttgctagct

SEQ ID NO: 46 - DNA, 22 bp, Homo sapiens
ccgcagacgtgtaaatgttcct

SEQ ID NO: 47 - DNA, 22 bp, Homo sapiens
cggcttgtcacatctgcaagta

SEQ ID NO: 48 - DNA, 22 bp, Homo sapiens
caaagtgggcacagatggtgtg

SEQ ID NO: 49 - DNA, 21 bp, Homo sapiens
ctgcttggattccagaaacgg

SEQ ID NO: 50 - protein, 241 aa, Homo sapiens
MDVLPMCSIFQELQIVHETGYFSALPSLEEYWQQTCLELERYLQSEPCYVSASEIKFDSQEDLWTKIILAREKKEESELKISSSPPEDTLISPSFCYNLETNSLNSDVSSESSDSSEELSPTAKFTSDPIGEVLVSSGKLSSSVTSTPPSSPELSREPSQLWGCVPGELPSPGKVRSGTSGKPGEKPYRCSWEGCEWRFARSDELTRHFRKHTGAKPFKCSHCDRCFSRSDHLGLHMKRHL

SEQ ID NO: 51 - protein, 237 aa, Homo sapiens
MDVLPMCSIFQELQIVHETGYFSALPSLEEYWQQTCLELERYLQSEPCYVSASEIKFDSQEDLWTKIILAREKKEESELKISSSPPEDTLISPSFCYNLETNSLNSDVSSESSDSSEELSPTAKFTSDPIGEVLVSSGKLSSSVTSTPPSSPELSREPSQLWGCVPGELPSPGKVRSGTSGKPGDKGNGDASPDGRRRVHRCHFNGCRKVYTKSSHLKAHQRTHTGVFPGLTTWACT

SEQ ID NO: 52 - DNA, 21 bp, Homo sapiens
gactacaggcgcccaccacca

SEQ ID NO: 53 - DNA, 24 bp, Homo sapiens
tgtgggactcccatacaattaaaa

SEQ ID NO: 54 - DNA, 22 bp, Homo sapiens
gccccgacttgtctctctacaa

SEQ ID NO: 55 - DNA, 23 bp, Homo sapiens
tggagatggggtctcactctttc

SEQ ID NO: 56 - DNA, 22 bp, Homo sapiens
gtcttagtgagccaccggcggg

SEQ ID NO: 57 - DNA, 21 bp, Homo sapiens
gttcacctgccggccacagcc

SEQ ID NO: 58 - DNA, 17 bp, Homo sapiens
gcggtacggggggcggt

SEQ ID NO: 59 - DNA, 20 bp, Homo sapiens
acgccgagcgagggcaggcg

SEQ ID NO: 60 - DNA, 18 bp, Homo sapiens
ggcttttctccttccctg